(19) United States
(12) Patent Application Publication
(10) Pub. No.: US 2013/0091759 A1
(43) Pub. Date: Apr. 18, 2013

(54) METHOD OF BIOBASED CHEMICAL PRODUCTION FROM CRUDE BIOGLYCERIN OF PLANT ORIGIN

(71) Applicants: John R. Peterson, Chardon, OH (US); Christopher M. Yost, Ayr (CA)

(72) Inventors: John R. Peterson, Chardon, OH (US); Christopher M. Yost, Ayr (CA)

(73) Assignee: THESIS CHEMISTRY, LLC, Mentor, OH (US)

(21) Appl. No.: 13/348,777

(22) Filed: Jan. 12, 2012

Related U.S. Application Data
(63) Continuation-in-part of application No. 13/271,925, filed on Oct. 12, 2011.

(51) Int. Cl. C10L 1/00; C07C 69/74; C07C 49/00; C07C 53/00; C07C 31/04; C07C 45/00; C07C 35/00; C07C 31/02; C07C 31/18; C07C 31/08; C07D 301/02; C07C 47/00

(52) U.S. Cl. 44/307; 549/518; 560/129; 562/512; 568/303; 568/420; 568/449; 568/700; 568/840; 568/852

(57) ABSTRACT

A method of production of value-added, biobased chemicals, derivative products, and/or purified glycerin from plant-based bioglycerin is described herein. A method of purification of plant-based bioglycerin is also described herein. The method of purification of plant-based bioglycerin described provides methods for desalinating, decolorizing, and/or concentrating plant-based bioglycerin for the production of biobased chemicals, derivative products, and/or purified glycerin.

This application is a continuation-in-part and claims priority from U.S. Ser. No. 13/271,925, titled METHOD OF BIOBASED CHEMICAL PRODUCTION FROM CRUDE BIOGLYCERIN, filed Oct. 12, 2011, which is incorporated herein by reference.

### I. BACKGROUND OF THE INVENTION

A. Field of Invention

The present invention is directed generally to a method of production of value-added, biobased chemicals, derivative products, and/or purified glycerin from plant-based bioglycerin. A method of purification of a crude plant-based bioglycerin is described herein, which provides methods for desalinating, decolorizing, and/or concentrating a plant-based bioglycerin for the production of biobased chemicals, derivative products, and/or purified glycerin.

B. Description of the Related Art

The world currently faces depletion of fossil fuels while demands for these fuels are ever increasing. Petrochemicals provide an energy source and a component of the majority of raw materials used in many industries. In fact, approximately 95% of all chemicals manufactured today are derived from petroleum. However, this heavy reliance upon fossil fuels is creating harm to the environment. The burning of these fossil fuels has led to the pollution of air, water and land, as well as global warming and climate changes. Through the use of fossil fuels, the environment has been harmed, perhaps irreparably, in an effort to meet the nearly insatiable demand for energy and manufactured products. Fossil fuels are a finite natural resource, with the depletion of readily available oil reserves across the globe; the supply chain has shifted to more complex and environmentally risky production technologies. A reduction and conservation of fossil fuels is clearly needed. Some alternatives to fossil fuels, like solar power, wind power, geothermal power, hydropower, and nuclear power, are used to a degree. However, a more efficient use of renewable resources is always being sought.

In particular, biofuels, which come from a renewable, carbonaceous source, are targeted to become one of these more efficient resources. In the demand for fossil fuels, biodiesel, a type of biofuel, has emerged as a potentially inexhaustible alternative to petroleum diesel, particularly during an oil crisis, a surge in crude oil prices, and/or political unrest in the oil producing regions of the world. This renewable and clean-burning diesel replacement is said to reduce our dependence on foreign petroleum and create new employment within the green industry.

Biodiesel is considered as an environmentally friendly, renewable transportation and heating fuel relative to petroleum diesel. Biodiesel can be made from plant-based triglycerides of agricultural plant or other plant origin. Agricultural plant oils and/or other plant-based oils may contain these plant-based triglycerides. Some of these agricultural plant oils may be soybean oil, corn oil, cottonseed oil, canola oil, rice bran oil, flax oil, sunflower oil, safflower oil, artichoke oil, sesame oil, and peanut oil. Some of these other plant-based oils may be castor oil, coconut oil, colza oil, false flax oil, hemp oil, mustard oil, palm oil, radish oil, rapeseed oil, tigernut oil, tung oil, copaiba oil, jatropha oil, jojoba oil, karanj oil, milk bush (pencil bush) oil, neem oil, olive oil, salicornia oil, and paradise oil. Based on this plant-based feedstock sources for biodiesel, agricultural plant oils and/or other plant-based oils, and waste streams of agricultural plant oils and/or other plant-based oils, may provide renewable means for replacing fossil fuels.

However, the production of biodiesel from plant-based feedstock sources does present a production by-product: a crude plant-based bioglycerin. Biodiesel from plant-based feedstock sources consists of mono-alkyl esters of long chain fatty acids that are formed by reaction of the triglyceride present in the plant-based oil with an alcohol. This process yields plant-based biodiesel through a hydrolysis and/or transesterification reaction during which the crude plant-based bioglycerin is cleaved from the plant-based triglyceride as a by-product. Thus, the process yields two products: plant-based biodiesel and a crude plant-based bioglycerin. The crude plant-based bioglycerin is formed in approximately 1 part to each 10 parts of the plant-based biodiesel. In the pure form, glycerin is a colorless, viscous liquid; however, the crude plant-based bioglycerin may be a yellowish to dark brown liquid. It may be a clear to a turbid liquid, or have a syrup-like consistency. The crude plant-based bioglycerin may contain significant amounts of particulate matter, dissolved inorganic salts, alcohol, water, unreacted fatty acids, and other impurities from the biodiesel process. Because of the high content of these impurities, which can range from about 5% to more than about 30%, uses for the crude plant-based bioglycerin are limited while escalating global biodiesel production is culminating in a market glut for this by-product. Additionally, varying purity levels of the crude plant-based bioglycerin due to different feedstock sources of the biodiesel, even among various plant-based feedstock sources, as well as different levels of in-process control among biodiesel producers, do not provide a uniform approach to treating the crude plant-based bioglycerin by-product. Even if the crude plant-based bioglycerin is treated, the purification of the crude plant-based bioglycerin historically has been too expensive and commercial implementation of a crude plant-based bioglycerin purification process is yet to prove economical at large scale.

Because the crude plant-based bioglycerin can be expensive to purify and market demand for the crude material is limited, it is often sold at a significant discount relative to the price of a petroleum-based glycerin. In lieu of a market outlet, the crude plant-based bioglycerin would quickly accumulate as an unwanted waste product of plant-based biodiesel production with associated disposal costs. Although this green process of creating biofuel is grounded upon the sustainable use of renewable resources, the process unfortunately generates a low-value by-product that diminishes the overall green value of biodiesel production. However, a purified plant-based bioglycerin from the production of this biofuel would provide an even greener process as well as become a potential additional revenue stream for biodiesel producers. Such a purified plant-based bioglycerin could compete and function as a green replacement to a petroleum-derived glycerin and/or serve as a renewable feedstock for the production of value-added, biobased chemicals, derivative products, and/or purified glycerin.

In the pure form, glycerin has many uses. It is used in the food and beverage industry as a humectant, sweetener, solvent, preservative, filler, emulsifier, and thickening agent. It also has several uses in the personal care and pharmaceutical industries where it functions as a lubricant, humectant, laxative, bacteriostat, moisturizer and pharmaceutical excipient. It is a well-known component of glycerin soaps. It also has applications in tobacco, polyether polyols, alkyd resins, paints, coatings, lubricants, textiles, paper, biological research, fabric softeners, cellophane, explosives, and epoxy resins. Purer forms of a plant-based bioglycerin also command a higher market value as compared to a less pure plant-based bioglycerin. Additionally, potential emerging applications for a plant-based bioglycerin include its conversion into commodity chemicals, like 1,2-propanediol and 1,3-propanediol, and into fine chemicals like epichlorohydrin, glycidyl ethers and glycidyl esters. Once implemented, these applications are expected to further improve global market demand for plant-based bioglycerin. Overall, a purified plant-based bioglycerin from plant-based biodiesel production could serve as a feedstock for production of value-added, biobased chemicals, derivative products, and/or purified glycerin, and as a means to reduce costs associated with waste stream disposal.

The present invention provides methods for purifying crude plant-based bioglycerin and converting crude plant-based bioglycerin and/or a purified plant-based bioglycerin into value-added, biobased chemicals, derivative products, and/or purified glycerin while minimizing waste products.

### II. SUMMARY OF THE INVENTION

Accordingly, it is an object of the invention to provide a method of biorefining. It may include the steps of providing crude plant-based bioglycerin and treating the crude plant-based bioglycerin through one or more steps of a crude plant-based bioglycerin purification process to provide a purified plant-based bioglycerin. Additionally, the crude plant-based bioglycerin may be provided from at least one plant-based triglyceride provided from soybean oil, corn oil, cottonseed oil, canola oil, rice bran oil, flax oil, sunflower oil, safflower oil, artichoke oil, sesame oil, peanut oil, castor oil, coconut oil, colza oil, false flax oil, hemp oil, mustard oil, palm oil, radish oil, rapeseed oil, tigernut oil, tung oil, copaiba oil, jatropha oil, jojoba oil, karanj oil, milk bush (pencil bush) oil, neem oil, olive oil, salicornia oil, and paradise oil. The method may further include the steps of producing at least one biobased chemical, derivative product, and/or purified glycerin from the crude plant-based bioglycerin and/or a purified plant-based glycerin.

One object of the present invention is that a purified plant-based bioglycerin may be produced from a by-product of plant-based biodiesel production.

Another object of the present invention is that the steps of treating the crude plant-based bioglycerin to provide a purified plant-based bioglycerin comprises at least one step of desalination treatment, decolorization treatment, and concentration treatment.

Yet another object of the present invention is that the desalination treatment provides a desalinated plant-based bioglycerin, the decolorization treatment provides a decolorized plant-based bioglycerin, and the concentration treatment provides a concentrated plant-based bioglycerin.

Still another object of the present invention is that the step of treating the crude plant-based bioglycerin by the desalination treatment to provide a desalinated plant-based bioglycerin may use an ion exchange treatment.

Yet another object of the present invention is that the step of treating the crude plant-based bioglycerin to provide a decolorized plant-based bioglycerin can use a decolorizing treatment process.

Still yet another object of the present invention is that the step of treating the crude plant-based bioglycerin to provide a concentrated plant-based bioglycerin may use a concentration treatment process.

Still another object of the present invention is that the steps of the desalination treatment, the decolorization treatment, and the concentration treatment for purification of the crude plant-based bioglycerin may be performed in any order.

A further object of the present invention is that one or more of the steps of the desalination treatment, the decolorization treatment, and the concentration treatment for purification of the crude plant-based bioglycerin may be repeated.

Yet another object of the present invention is that one or more of the steps of the desalination treatment, the decolorization treatment, and the concentration treatment for purification of the crude plant-based bioglycerin can be skipped.

Still yet another object of the present invention is that the desalination treatment step of the crude plant-based bioglycerin purification process may be performed under batch or continuous flow conditions.

According to one embodiment of the present invention, a solvent can be added, recovered and recycled during the desalination treatment step of the crude plant-based bioglycerin purification process.

According to another embodiment of the present invention, the ion exchange resins may be regenerated and recycled during the desalination treatment step of the crude plant-based bioglycerin purification process.

According to still another embodiment of the present invention, the desalination treatment step of the crude plant-based bioglycerin purification process can be low energy demanding.

According to still yet another embodiment of the present invention, the desalination treatment step of the crude plant-based bioglycerin purification process can recover salt, which is useful for commercial de-icing or lowering the freezing point of solutions.

According to still yet another embodiment of the present invention, the desalination treatment step of the crude plant-based bioglycerin purification process produces water that may be recovered and reused.

Still another object of the present invention is that the decolorization treatment step of the crude plant-based bioglycerin purification process may be performed under batch or continuous flow conditions.

According to one embodiment of the present invention, a solvent may be added, recovered and recycled during the decolorization treatment step of the crude plant-based bioglycerin purification process.

According to another embodiment of the present invention, the decolorization treatment step of the crude plant-based bioglycerin purification process may use an activated charcoal.

According to still another embodiment of the present invention, the decolorization treatment step of the crude plant-based bioglycerin purification process can be low energy demanding.

According to still yet another embodiment of the present invention, can be the recovery and recycle of the activated charcoal used in the decolorization treatment step of the crude plant-based bioglycerin purification process.

Still another object of the present invention is that the concentration treatment step of the crude plant-based bioglycerin purification process can be performed under batch or continuous flow conditions.

According to one embodiment of the present invention, the concentration treatment step of the crude plant-based bioglycerin purification process can be performed at reduced pressure and modest temperature.

According to another embodiment of the present invention, the concentration treatment step of the crude plant-based bioglycerin purification process can be low energy demanding.

According to yet another embodiment of the present invention is the recovery and recycle of water and solvent in the concentration treatment step of the crude plant-based bioglycerin purification process.

Another object of the present invention is that the yield recovery of a purified plant-based bioglycerin from the crude plant-based bioglycerin purification process may be greater than 80% of the theoretical yield amount of the plant-based bioglycerin from plant-based biodiesel production.

Yet another object of the present invention is the yield recovery of a purified plant-based bioglycerin from the crude plant-based bioglycerin purification process can be greater than 90% of the theoretical yield amount of the plant-based bioglycerin from plant-based biodiesel production.

Another object of the present invention is that the weight of a purified plant-based bioglycerin from the crude plant-based bioglycerin purification process may be greater than 60% of the weight of the crude plant-based bioglycerin.

Still another object of the present invention is that the weight of a purified plant-based bioglycerin from the crude plant-based bioglycerin purification process may be greater than 80% of the weight of the crude plant-based bioglycerin.

Still yet another object of the present invention is that the weight of a purified plant-based bioglycerin from the crude plant-based bioglycerin purification process can be greater than 90% of the weight of the crude plant-based bioglycerin.

Still yet another object of the present invention can be the steps of producing one or more of the biobased chemicals, derivative products, and/or purified glycerin from the crude plant-based bioglycerin and/or a purified plant-based bioglycerin.

According to one embodiment of the present invention, purified plant-based bioglycerins of different purities can produce one or more of the biobased chemicals, derivative products, and/or purified glycerin.

According to another embodiment of the present invention, the production of the biobased chemicals and/or derivative products from the crude plant-based bioglycerin and/or a purified plant-based bioglycerin may take place by a chemical process.

According to still another embodiment of the present invention, the production of the biobased chemicals and/or derivative products from the crude plant-based bioglycerin and/or a purified plant-based bioglycerin may take place by a biological process.

According to yet another embodiment of the present invention, the production of the biobased chemicals and/or derivative products from the crude plant-based bioglycerin and/or a purified plant-based bioglycerin may take place by a catalytic process.

According to still yet another embodiment of the present invention, the production of the biobased chemicals and/or derivative products from the crude plant-based bioglycerin and/or a purified plant-based bioglycerin may take place by a pyrolytic process.

According to yet another embodiment of the present invention, the production of the biobased chemicals and/or derivative products from the crude plant-based bioglycerin and/or a purified plant-based bioglycerin can involve one or more chemical, biological, catalytic, and pyrolytic processes.

Further, another object of the present invention can be functionalizing the crude plant-based bioglycerin and/or a purified plant-based bioglycerin to form a functionalized plant-based bioglycerin product prior to the production of the biobased chemicals and/or derivative products.

According to another aspect, the present invention can provide for the production of a plurality of the biobased chemicals and/or derivative products from the crude plant-based bioglycerin and/or a purified plant-based bioglycerin comprising but not limited to purified glycerin, glycerin derivatives, C1-C3 alcohols, C2/C3 diols, C1-C3 aldehydes/ketones, C1-C3 carboxylic acids, C1-C3 esters of C1-C3 carboxylic acids, C5/C6 polyols, polyol derivatives, glycidol, glycidyl derivatives, glyceraldehyde, glyceraldehyde derivatives, and epihalohydrins.

According to yet another aspect, the present invention can provide for the production of a plurality of biobased chemicals and/or derivative products from the crude plant-based bioglycerin and/or a purified plant-based bioglycerin, comprising but not limited to purified glycerin, methanol, ethanol, n-propanol, isopropanol, allyl alcohol, propargyl alcohol, ethylene glycol, 1,2-propanediol, 1,3-propanediol, formaldehyde, acetaldehyde, propionaldehyde, glyoxal, acrolein, acetone, 1-hydroxyacetone, 1,3-dihydroxyacetone, formic acid, acetic acid, glycolic acid, glyoxylic acid, oxalic acid, propionic acid, lactic acid, 2,3-dihydroxypropionic acid, pyruvic acid, acrylic acid, malonic acid, hydroxymalonic acid, methyl formate, methyl acetate, methyl glycolate, methyl glyoxylate, dimethyl oxalate, methyl propionate, methyl lactate, methyl 2,3-dihydroxypropionate, methyl pyruvate, methyl acrylate, dimethyl malonate, dimethyl hydroxymalonate, ethyl formate, ethyl acetate, ethyl glycolate, ethyl glyoxylate, diethyl oxalate, ethyl propionate, ethyl lactate, ethyl 2,3-dihydroxypropionate, ethyl pyruvate, ethyl acrylate, diethyl malonate, diethyl hydroxymalonate, n-propyl formate, n-propyl acetate, n-propyl glycolate, n-propyl glyoxylate, di-n-propyl oxalate, n-propyl propionate, n-propyl lactate, n-propyl 2,3-dihydroxypropionate, n-propyl pyruvate, n-propyl acrylate, di-n-propyl malonate, di-n-propyl hydroxymalonate, isopropyl formate, isopropyl acetate, isopropyl glycolate, isopropyl glyoxylate, diisopropyl oxalate, isopropyl propionate, isopropyl lactate, isopropyl 2,3-dihydroxypropionate, isopropyl pyruvate, isopropyl acrylate, diisopropyl malonate, diisopropyl hydroxymalonate, allyl formate, allyl acetate, allyl glycolate, allyl glyoxylate, diallyl oxalate, allyl propionate, allyl lactate, allyl 2,3-dihydroxypropionate, allyl pyruvate, allyl acrylate, diallyl malonate, diallyl hydroxymalonate, glycerol formal, 4-(hydroxymethyl)-1,3-dioxolan-2-one, 4-methyl-1,3-dioxolane, (2,2-dimethyl-1,3-dioxolan-4-yl)methanol, 1,4-dioxaspiro[4.5]decane-2-methanol, glyceraldehyde, 2,2-dimethyl-1,3-dioxolane-4-carbaldehyde, 1,4-dioxaspiro[4.5]decane-2-carbaldehyde, glycidol, glycidyl methyl ether, glycidyl ethyl ether, glycidyl n-propyl ether, glycidyl isopropyl ether, glycidyl n-butyl ether, glycidyl isobutyl ether, glycidyl sec-butyl ether, glycidyl tert-butyl ether, glycidyl allyl ether, glycidyl propargyl ether, glycidyl hexadecyl ether, glycidyl octyl/decyl ether, glycidyl phenyl ether, glycidyl benzyl ether, glycidyl formate, glycidyl acetate, glycidyl propionate, glycidyl isopropionate, glycidyl n-butyrate, glycidyl isobutyrate, glycidyl sec-butyrate, glycidyl acrylate, glycidyl methacrylate, diglycidyl 1,2-cyclohexanedicarboxylate, glycidyl benzoate, glycidyl 4-nitrobenzoate, epichlorohydrin, epibromohydrin, ribitol, arabitol, xylitol, mannitol, sorbitol, galactitol, allitol, iditol, and bis-(2,2-dimethyl-(1,3)dioxolan-4-yl methanol.

Still yet another object of the present invention is the plurality of biobased chemicals and/or derivative products produced from the crude plant-based bioglycerin and/or a purified plant-based bioglycerin comprises at least one of achiral, racemic, and optically pure products.

Still another object of the present invention is that at least one of the biobased chemicals and/or derivative products produced from the crude plant-based bioglycerin and/or a purified plant-based bioglycerin can be used in the production of other chemicals, materials, and products.

Another object of the present invention is at least one of the biobased chemicals and/or derivative products produced from the crude plant-based bioglycerin and/or a purified plant-based bioglycerin comprises at least one of commodity chemicals, fine chemicals, and/or specialty chemicals.

Yet another object of the present invention is that it can provide a method of biorefining, comprising the steps of providing a crude plant-based bioglycerin, treating the crude plant-based bioglycerin by one or more of the desalination treatment, the decolorization treatment, and the concentration treatment steps to provide a purified plant-based bioglycerin, and producing a plurality of biobased chemicals, derivative products and/or purified glycerin from the crude plant-based bioglycerin and/or a purified plant-based bioglycerin.

Still another object of the present invention is that it can provide a method of biorefining. It may include the steps of providing a crude plant-based bioglycerin and treating the crude plant-based bioglycerin to provide a purified plant-based bioglycerin. The method may further include recovering and using the salts, water, and alcohol contaminating the crude plant-based bioglycerin from the plant-based biodiesel production process.

Yet another object of the present invention is that it can provide a method of biorefining. It may include the steps of providing a crude plant-based bioglycerin and treating the crude plant-based bioglycerin to provide a purified plant-based bioglycerin. The method may further include recovering and using any solvents used in the purification of the crude plant-based bioglycerin.

Still yet another object of the present invention is that it may provide a method of providing a crude plant-based bioglycerin and treating the crude plant-based bioglycerin to provide a purified plant-based bioglycerin where the waste product from the crude plant-based bioglycerin purification process can be used to produce energy.

Further, another object of the present invention can be to provide a method for biorefining that is easy to implement and use.

Still other benefits and advantages of the invention will become apparent to those skilled in the art to which it pertains upon a reading and understanding of the following detailed specification.

### III. BRIEF DESCRIPTION OF THE DRAWINGS

The invention may take physical form in certain parts and arrangement of parts, embodiments of which will be described in detail in this specification and illustrated in the accompanying drawings which form a part hereof, and wherein:

FIG. 1 is a flow diagram schematically illustrating the present invention.

FIG. 2 is a flow diagram schematically illustrating another aspect of the present invention.

FIG. 3 is a flow diagram schematically illustrating another aspect of the present invention.

FIG. 4 is a flow diagram schematically illustrating another aspect of the present invention.

FIG. 5 is a flow diagram schematically illustrating another aspect of the present invention.

FIG. 6 is a flow diagram schematically illustrating another aspect of the present invention.

FIG. 7 is a flow diagram schematically illustrating another aspect of the present invention.

FIG. 8 is a flow diagram schematically illustrating another aspect of the present invention.

FIG. 9 is a flow diagram schematically illustrating another aspect of the present invention.

FIG. 10 is a flow diagram schematically illustrating another aspect of the present invention.

FIG. 11 is a flow diagram schematically illustrating another aspect of the present invention.

FIG. 12 is a flow diagram schematically illustrating another aspect of the present invention.

### IV. DETAILED DESCRIPTION OF THE INVENTION

Referring now to the drawings wherein the showings are for purposes of illustrating embodiments of the invention only and not for purposes of limiting the same. Relative language used herein is best understood with reference to the drawings, in which like numerals are used to identify like or similar items.

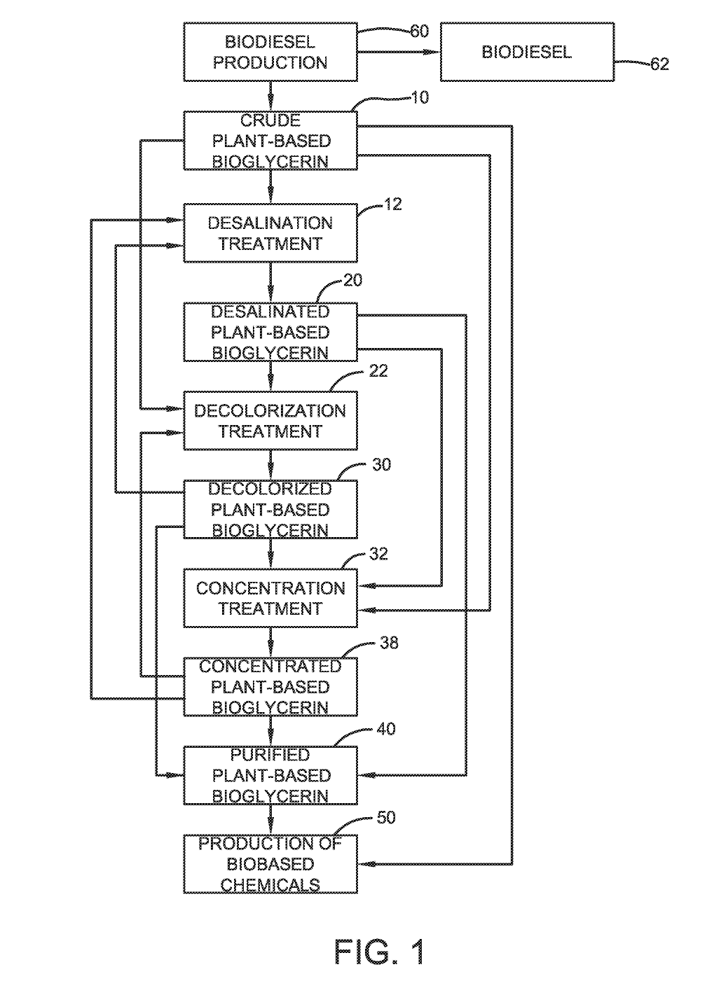

FIG. 1 shows the overall process of converting a crude plant-based bioglycerin 10 into a purified plant-based bioglycerin 40, and further in the production of biobased chemicals 50. It is a summary of the multiple pathways to process and use the crude plant-based bioglycerin 10 as a renewable, carbonaceous material for the production of biobased chemicals 50.

The crude plant-based bioglycerin 10 is a by-product of biodiesel production 60, through the hydrolysis and/or transesterification process used in the manufacture of biodiesel 62. Biodiesel production 60 from plant-based feedstock sources yields mostly biodiesel 62, with roughly 10% of the product mass being a crude plant-based bioglycerin 10. Escalating biodiesel production 60 across the globe is generating large quantities of crude plant-based bioglycerin 10 that could be used in the production of a purified plant-based bioglycerin 40 and/or the production of biobased chemicals 50. Additionally, the crude plant-based bioglycerin 10 may come from various sources. The crude plant-based bioglycerin 10 may be provided from at least one plant-based triglyceride of soybean oil, corn oil, cottonseed oil, canola oil, rice bran oil, flax oil, sunflower oil, safflower oil, artichoke oil, sesame oil, peanut oil, castor oil, coconut oil, colza oil, false flax oil, hemp oil, mustard oil, palm oil, radish oil, rapeseed oil, tigernut oil, tung oil, copaiba oil, jatropha oil, jojoba oil, karanj oil, milk bush (pencil bush) oil, neem oil, olive oil, salicornia oil, and paradise oil.

The crude plant-based bioglycerin 10 can contain several impurities from the hydrolysis and/or transesterification process used in the manufacture of biodiesel 62. Such impurities can include water and an alcohol like methanol or ethanol, with methanol the more typical alcohol impurity. The presence of an alcohol in the crude plant-based bioglycerin 10 may be due to the fact that an excess of this alcohol can be used to drive the hydrolysis and/or transesterification process of biodiesel production 60 to completion. Also, different biodiesel manufacturers may recover the excess alcohol to varying extents, leading to an inconsistent crude plant-based bioglycerin 10. In addition to the alcohol and water impurities, the crude plant-based bioglycerin 10 may contain dissolved salts, like sodium chloride, sodium sulfate, potassium chloride, potassium sulfate, calcium chloride, and calcium sulfate. These salts may arise from neutralization of the transesterification and/or hydrolysis process used in biodiesel production 60. Furthermore, the crude plant-based bioglycerin 10 may contain residual fatty acids and other impurities leading to color. These impurities may result from either an incomplete process of biodiesel production 60 or from contaminants in the plant-based feedstock source entering the refinery. The levels of water and alcohol contamination in the crude plant-based bioglycerin 10 may be controlled by evaporation/distillation or by implementing tighter control of the biodiesel processing parameters. However, the salts, which may amount to about 4-10% of the total impurities in the crude plant-based bioglycerin 10, can be more challenging to remove. Further these salts may impede transformations of the crude plant-based bioglycerin 10 into the purified plant-based bioglycerin 40 and/or the production of biobased chemicals 50.

Because of these impurities, there may be a limited market demand for the crude plant-based bioglycerin 10 and the market that does exist often may command a price as low as 1/10 that of petroleum-derived glycerin. The reason for this limited demand may be that these impurities, and in particular the salts, may severely hamper or restrict uses of the crude plant-based bioglycerin 10. Historically, the purification of the crude plant-based bioglycerin 10, and in particular the removal of the salt impurities, has proven too expensive for commercial implementation. For example, the purification of the crude plant-based bioglycerin 10 by distillation can be a very energy demanding process because the boiling point of glycerin is 290° C. (554° F.). However, the purification process illustrated in FIG. 1 provides a low energy, self-contained process that can remove both the salts and other impurities from the crude plant-based bioglycerin 10. The purification process shown in FIG. 1 can operate as a stand-alone biorefinery receiving the crude plant-based bioglycerin 10 for production of the purified plant-based bioglycerin 40 and/or the production of biobased chemicals 50, or it can provide an additional on-site option to a biodiesel manufacturer for waste stream reduction and/or value-added products production.

During biodiesel production 60, the crude plant-based bioglycerin 10 may have an inconsistent appearance or impurity profile from batch to batch or from producer to producer. These differences in appearance or impurity profile may be associated with the characteristics of different plant-based feedstock sources coming into these biodiesel facilities, and/or differences in the processes and manufacturing controls used across different biodiesel facilities. The crude plant-based bioglycerin 10 obtained from biodiesel production 60 can appear as a golden or slightly yellow liquid, or a dark brown substance that may have a liquid to a syrup-like character. The crude plant-based bioglycerin 10 can be translucent or turbid in appearance. Depending on the condition of the crude plant-based bioglycerin 10, several steps within the process of FIG. 1 can be carried out to produce a purified plant-based bioglycerin 40 and/or the production of biobased chemicals 50. These processes can be tailored to meet the end product requirements for a purified plant-based bioglycerin 40 and/or the raw material specification requirements for the production of biobased chemicals 50 from a purified plant-based bioglycerin 40.

Depending on the condition of the crude plant-based bioglycerin 10, it may need to be subjected to the desalination treatment 12, the decolorization treatment 22, and/or the concentration treatment 32. These processing treatments required for purifying the crude plant-based bioglycerin 10 depend on the end product requirements for the purified plant-based bioglycerin 40 and/or the raw material specification requirements for the production of biobased chemicals 50 from a purified plant-based bioglycerin 40.

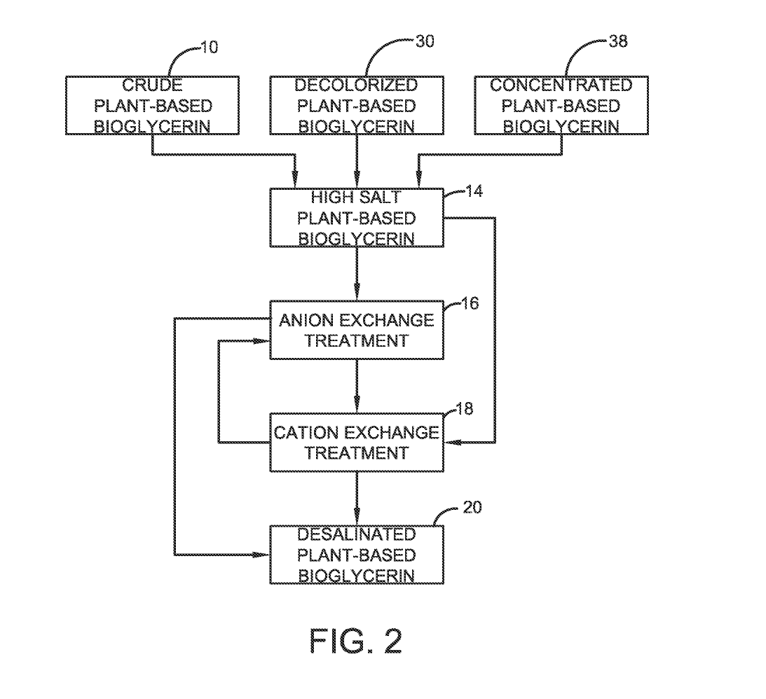

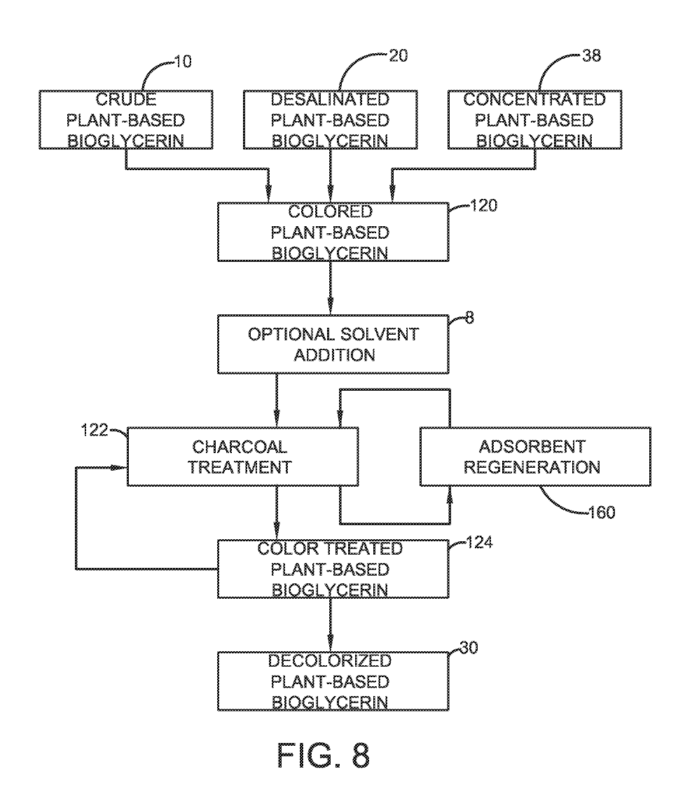

For instance, if the crude plant-based bioglycerin 10 from the biodiesel production 60 requires desalination, it may undergo the desalination treatment 12 to become a desalinated plant-based bioglycerin 20. Because these salt impurities can interfere with the purified plant-based bioglycerin 40 in the production of biobased chemicals 50, the desalination treatment 12 step is used to remove these impurities in order to provide a desalinated plant-based bioglycerin 20. The desalination treatment 12 step is further detailed in FIG. 2. The desalinated plant-based bioglycerin 20 may then go through the decolorization treatment 22 to obtain a decolorized plant-based bioglycerin 30 if a lighter colored material is needed. If the crude plant-based bioglycerin 10 has not been desalinated, it may also go through the decolorization treatment 22 if required. The decolorization treatment 22 may reduce the level of residual fatty acids and other colored impurities in the material. The decolorization treatment 22 step is further detailed in FIG. 8.

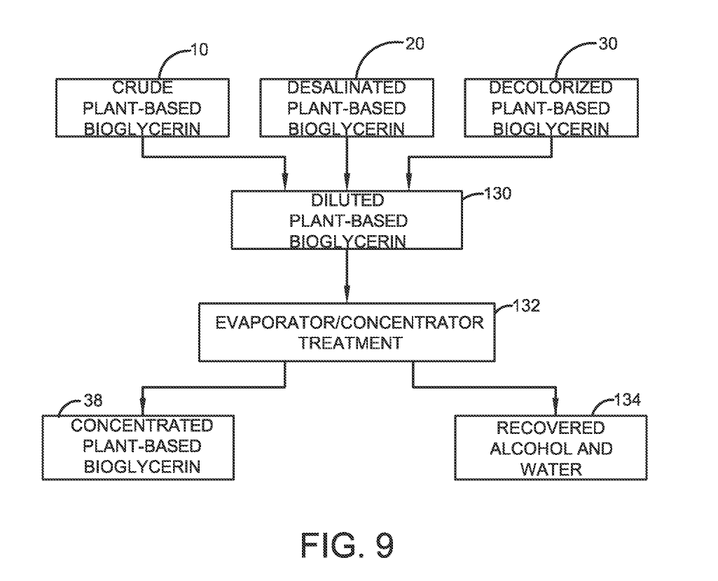

The crude plant-based bioglycerin 10, the desalinated plant-based bioglycerin 20, and/or the decolorized plant-based bioglycerin 30 may then undergo the concentration treatment 32 wherein further the alcohol and water impurities are removed to provide a concentrated plant-based bioglycerin 38. The concentration treatment 32 step is detailed in FIG. 9. After the concentration treatment 32 step is complete, a purified plant-based bioglycerin 40 can be produced. If desired, the purified plant-based bioglycerin 40 can then be transformed into commodity chemicals, fine chemicals, and/or specialty chemicals through a production of biobased chemicals 50 step or it can be further purified. For this process, the purification of the crude plant-based bioglycerin 10 does not have to begin with the desalination treatment 12. The purification process may start with the desalination treatment 12, the decolorization treatment 22, or the concentration treatment 32, or it can proceed directly to the production of biobased chemicals 50.

Within the overall process of converting the crude plant-based bioglycerin 10 into a purified plant-based bioglycerin 40, and/or potentially further into production of biobased chemical products 50, several steps may be omitted if the crude plant-based bioglycerin 10 does not require the desalination treatment 12, the decolorization treatment 22, and/or the concentration treatment 32 step to achieve the end product specification for the purified plant-based bioglycerin 40 and/or for the production of biobased chemicals 50. Depending on the condition of the intermediate plant-based bioglycerin product during any step of the purification process shown in FIG. 1, a determination of whether the material needs to undergo a further treatment can be made. For example, the crude plant-based bioglycerin 10 may be subjected to the desalination treatment 12 to remove the salt impurities. If the crude plant-based bioglycerin 10 does not require desalination, the desalination treatment 12 can be skipped and crude plant-based bioglycerin 10 may then be sent to the decolorization treatment 22 to improve its color. If desalination is required at this point, the decolorized plant-based bioglycerin 30 can then be subjected to the desalination treatment 12 to remove the salt impurities. If no desalination is needed, then the decolorized plant-based bioglycerin 30 can either go through the concentration treatment 32, or it can be directly converted into the purified plant-based bioglycerin 40. Alternatively, the crude plant-based bioglycerin 10 may directly be sent to the concentration treatment 32 for production of a concentrated plant-based bioglycerin 38, which may be converted into either the purified plant-based bioglycerin 40 and/or sent for the production of biobased chemicals 50 if neither desalination nor decolorization is required. In other instances, the crude plant-based bioglycerin 10 may omit the desalination treatment 12, the decolorization treatment 22, and the concentration treatment 32 and be directly converted into commodity chemicals, fine chemicals, and/or specialty chemicals with the production of biobased chemicals 50 step.

After the desalination treatment 12, the desalinated plant-based bioglycerin 20 may be sufficiently treated to become a purified plant-based bioglycerin 40 if the specification requirements are met for a purified plant-based bioglycerin 40 and/or for the production of biobased chemicals 50. Alternatively, if additional processes are needed for the desalinated plant-based bioglycerin 20 but not the decolorization treatment 22, then the desalinated plant-based bioglycerin 20 may be sent to the concentration treatment 32 where it becomes a concentrated plant-based bioglycerin 38, which can be used for the conversion to a purified plant-based bioglycerin 40 and/or sent for the production of biobased chemicals 50.

Additionally, the concentrated plant-based bioglycerin 38 may be processed to a purified plant-based bioglycerin 40, or undergo either the desalination treatment 12 or the decolorization treatment 22 before it can be used for the conversion to a purified plant-based bioglycerin 40 and/or sent for the production of biobased chemicals 50.

One detail to note during these processes is that the summary of the pathway shown in FIG. 1 may be changed to include a different order for the processes. This is designated by the additional arrows that demonstrate that the final product may not be dependent upon a particular order of the treatments, but rather which treatments can be applied and what processes may be required for the desired end product. This difference in order may be needed due to processing limitations or discoveries with respect to what may be required to meet the specifications of the end product or intended use as a purified plant-based bioglycerin 40 and/or sent for the production of biobased chemicals 50. In other words, the order of treatments provided in FIG. 1 does not have to be strictly followed. The desalination treatment 12, the decolorization treatment 22, and the concentration treatment 32 may occur in any order depending on the end product requirements for the purified plant-based bioglycerin 40 and/or the production of biobased chemicals 50. The order for the treatments may also depend upon logistics of the processing facility.

Also, any of the process treatment steps like the desalination treatment 12, the decolorization treatment 22, or the concentration treatment 32, may be repeated to provide the requirements for a purified plant-based bioglycerin 40 and/or the production of biobased chemicals 50.

Furthermore, any of the process treatment steps like the desalination treatment 12, the decolorization treatment 22, or the concentration treatment 32, may be conducted under batch or flow conditions for the production of a purified plant-based bioglycerin 40 and/or the production of biobased chemicals 50.

The processing outlined in FIG. 1 can also address problems with processing the crude plant-based bioglycerin 10 without the need to invest large amounts of capital in expensive processing equipment to purify this by-product of biodiesel production 60.

The processing outlined in FIG. 1 can further avoid the high costs of purifying the crude plant-based bioglycerin 10 by conventional means in that the process in FIG. 1 can be low energy and self-contained.

FIG. 2 illustrates an overview of the process for the desalination treatment 12 in which the high salt plant-based bioglycerin 14 may be transformed into a desalinated plant-based bioglycerin 20. This desalination process may occur through ion exchange to remove the salt impurities. The ion exchange treatment or process can be a two-stage process consisting of both the anion exchange treatment 16 and the cation exchange treatment 18. This two-step, ion exchange treatment can utilize both anion exchange resins and cation exchange resins to purify the high salt plant-based bioglycerin 14 by acting to exchange the ions that contribute to the salt impurities of the high salt plant-based bioglycerin 14. Anions are atoms or groups of atoms that have gained electrons, and are therefore negatively charged. Cations are atoms or groups that have lost an electron to become positively charged. Together, anions and cations form salts like sodium chloride, sodium sulfate, potassium chloride, potassium sulfate, calcium chloride, and calcium sulfate. Anion exchange resins and cation exchange resins can be both selective and versatile, where specific types of ions can be removed from a material depending on the specific anion exchange treatment 16 and the cation exchange treatment 18 chosen.

First, the high salt plant-based bioglycerin 14 may be received. The high salt plant-based bioglycerin 14 can originate from a crude plant-based bioglycerin 10, a decolorized plant-based bioglycerin 30 and/or a concentrated plant-based bioglycerin 38. The high salt plant-based bioglycerin 14 may then undergo the anion exchange treatment 16. The anion exchange treatment 16 step can serve to reduce or remove the anionic impurities present in the high salt plant-based bioglycerin 14 by use of an anion exchange resin, which exchanges the negatively charged ions of the salt impurities in the high salt plant-based bioglycerin 14 with the counterion bound to the resin. For instance, this anion exchange treatment 16 may remove halide, sulfate, and other anions first from the high salt plant-based bioglycerin 14 and replace those anions with hydroxide anions. Through the anion exchange treatment 16, the anionic components of the salt impurities can be removed from the high salt plant-based bioglycerin 14. After the anion exchange treatment 16 step is completed, the cation exchange treatment 18 step may then occur to reduce and replace the cations from the salt impurities present in the high salt plant-based bioglycerin 14 with the counterion bound to the cation exchange resin, typically a proton. Through the cation exchange treatment 18, the cationic components of the salt impurities may be reduced and removed from the high salt plant-based bioglycerin 14. For example, this cation exchange treatment 18 may remove sodium, potassium, calcium, and other cations and replace those cations with protons. Therefore, through both anion and cation exchange treatment steps, the high salt plant-based bioglycerin 14 can be reduced in levels of both positively and negatively charged ionic salt impurities, and the desalinated plant-based bioglycerin 20 may now be formed. The desalinated plant-based bioglycerin 20 may then go through one or more additional treatments of decolorization, concentration, and/or transfer to the production of plant-based biobased chemicals 50 as described in FIG. 1. In the course of the desalination treatment 12, water can be produced as a by-product through a combination of the hydroxide anions derived from the anion exchange treatment 16 step with the protons derived from the cation exchange treatment 18 step. To reduce waste streams, this water may be recovered and reused in the desalination treatment 12.

Although the desalination treatment 12 in which the high salt plant-based bioglycerin 14 is transformed into a desalinated plant-based bioglycerin 20 can be achieved by first subjecting the high salt plant-based bioglycerin 14 to the anion exchange treatment 16 step and following that step with the cation exchange treatment 18 step, the process is not limited to this order of ion exchange treatments. Instead, the high salt plant-based bioglycerin 14 can first undergo the cation exchange treatment 18, and then followed by the anion exchange treatment 16. In other words, either ion exchange treatment can be used first.

Alternatively, the high salt plant-based bioglycerin 14 can undergo only one of the exchange treatments, either the anion exchange treatment 16 or the cation exchange treatment 18. Depending on the condition of the high salt plant-based bioglycerin 14 or the conditions required for the desalinated plant-based bioglycerin 20, the anion exchange treatment 16 or the cation exchange treatment 18 can be omitted.

Alternatively, an amphoteric exchange treatment could be used instead wherein both the anion exchange treatment 16 and the cation exchange treatment 18 occur at once. This type of amphoteric exchanger will exchange both cations and anions simultaneously. Instead of completing two different steps where the anion exchange treatment 16 step and the cation exchange treatment 18 step are separate, a process where all of the ion exchanging can occur in a condensed step may also be used.

Moreover in the course of the desalination treatment 12, each of the steps of the anion exchange treatment 16 and the cation exchange treatment 18 may be conducted more than one time. Repeating the anion exchange treatment 16 step and/or the cation exchange treatment 18 step can allow for applications wherein the levels of dissolved salts in the desalinated plant-based bioglycerin 20 may be further reduced, especially if required for certain specifications of intended product use.

The reduction in levels of both positively and negatively charged ions in the desalination treatment 12 may lead to the formation of a desalinated plant-based bioglycerin 20 since the salt impurities are reduced or removed by the ion exchange treatment or process. With the desalination treatment 12 of the high salt plant-based bioglycerin 14, both the possibility of creating value-added products and the prevention of a costly waste stream may provide incentives for utilizing the desalination treatment 12 process.

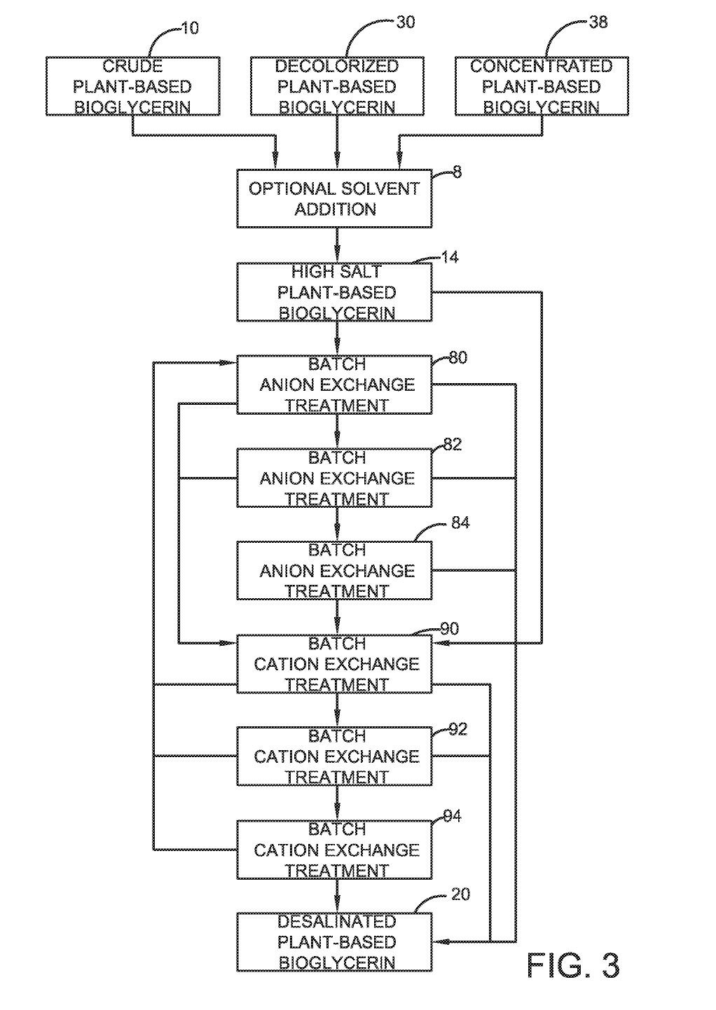

In FIG. 3, a detailed batch purification method for the desalination treatment 12 of the high salt plant-based bioglycerin 14 is shown. During this batch process, the high salt plant-based bioglycerin 14 can be converted into a desalinated plant-based bioglycerin 20. The ion exchange treatment or process may be done through multiple batch anion exchange and cation exchange treatments. These anion and cation exchange treatments typically employ an ion exchange resin to remove the negatively charged and positively charged ions that are present in the salt impurities of the high salt plant-based bioglycerin 14.

Ion exchange resins are classified as cation exchangers, on which positively charged mobile ions are available for exchange, and anion exchangers, on which the exchangeable ions are negatively charged. Both anion exchange resins and cation exchange resins may be produced from the same basic organic polymers. These resin types differ in the ionic functional group attached to the organic polymer network. It is this ionic functional group that determines the chemical behaviour of the resin. Ion exchange resins can be broadly classified as strong or weak acid cation exchangers, or strong or weak base anion exchangers. Ion exchange resins are insoluble substances containing loosely bound counterions that are able to be exchanged with other ions in solutions that come into contact with the resin. These exchanges take place without any physical alteration to the ion exchange material other than the exchange of the loosely bound counterions.

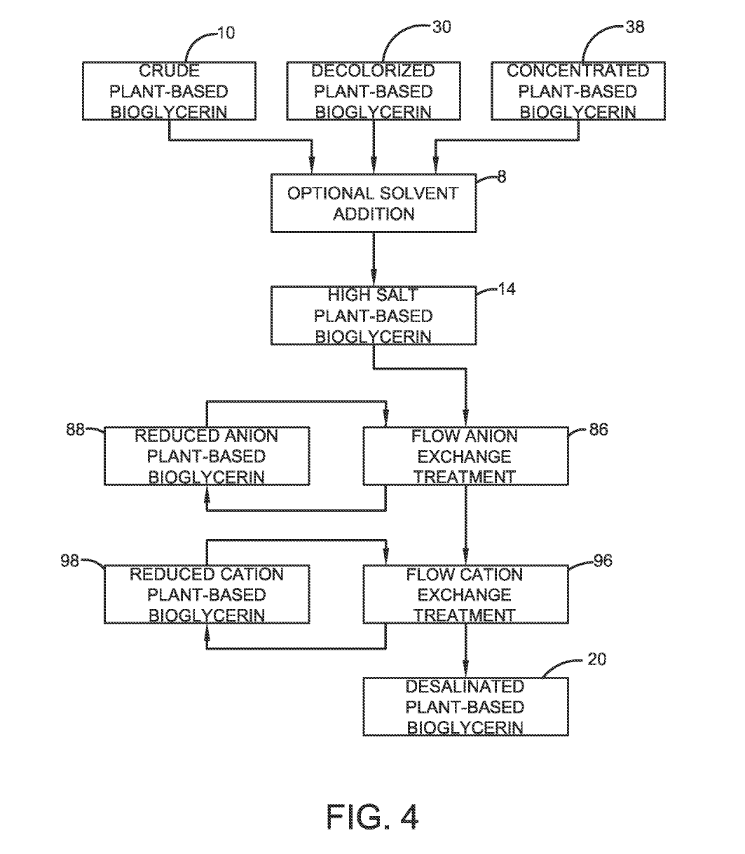

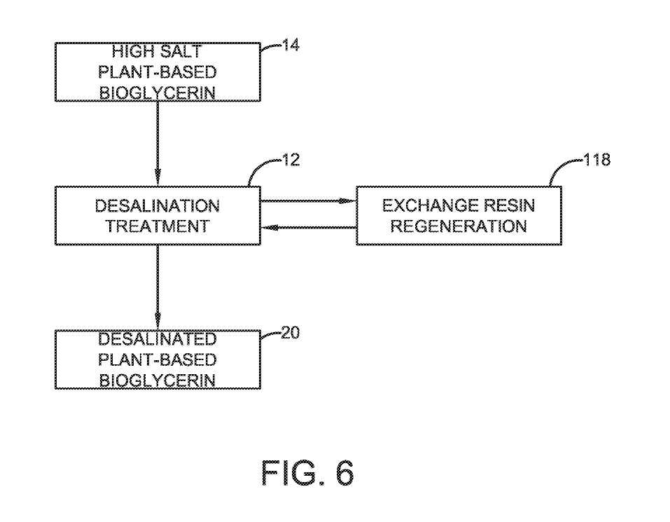

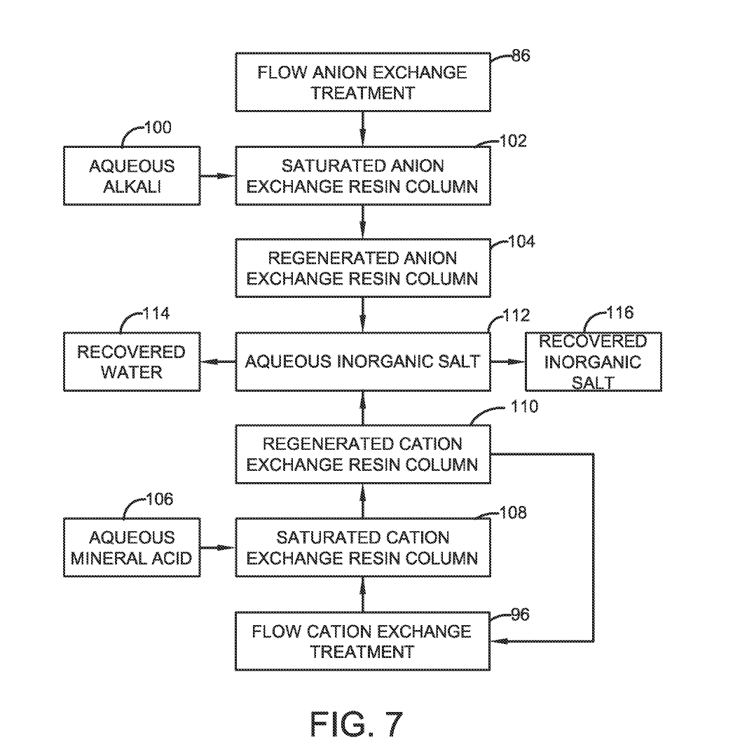

For the anion exchange treatment 16 and the cation exchange treatment 18 of the high salt plant-based bioglycerin 14 shown in FIG. 2, two different purification methods can be used: batch purification and continuous flow purification. In both instances, the high salt plant-based bioglycerin 14 would be subjected to ion exchange resins. Batch purification allows for purification in discrete batches. Batch purification is especially advantageous where different end products are needed. Continuous flow purification provides processing in a continuous flow, and allows for an increased production of a particular end product. A batch purification method is shown in FIG. 3. A continuous flow purification method is outlined in FIG. 4. There are, however, different modifications that must be considered in determining which purification method to use. In the batch purification method, the ion exchange resin is isolated by filtration before regeneration. This regeneration process for the batch purification method is illustrated generally in FIG. 6. Unlike the batch purification method, the continuous flow purification method regenerates the ion exchange resin within a column, as shown in FIG. 7. No matter which method is utilized, either method will provide the desalinated plant-based bioglycerin 20.

Returning now to FIG. 3, the high salt plant-based bioglycerin 14 can be received in the batch purification method for the desalination treatment 12. The high salt plant-based bioglycerin 14 may originate from a crude plant-based bioglycerin 10, a decolorized plant-based bioglycerin 30, and/or a concentrated plant-based bioglycerin 38. As the crude plant-based bioglycerin 10, the decolorized plant-based bioglycerin 30, and/or the concentrated plant-based bioglycerin 38 may be brought together as the high salt plant-based bioglycerin 14; an optional solvent addition 8 can be done. This optional solvent addition 8 can be water, an alcohol, an alcohol/water mixture, or another solvent in which the high salt plant-based bioglycerin 14 may be miscible. The optional solvent addition 8 may typically be an alcohol like methanol, but it may also be ethanol. This optional solvent addition 8 can serve to reduce the viscosity of the high salt plant-based bioglycerin 14 and help enhance recovery of the desalinated plant-based bioglycerin 20 from the ion exchange resins. Solvents used in the optional solvent addition 8 may be recovered in the concentration treatment 32, as shown in FIG. 1.

For FIG. 3, the high salt plant-based bioglycerin 14 may be subjected to multiple treatments with both anionic and cationic ion exchangers in order to produce the desalinated plant-based bioglycerin 20. The flow path for the ion exchange treatments can depend upon the anion and cation level specifications for production of either the desalinated plant-based bioglycerin 20 or the purified plant-based bioglycerin 40 for the production of biobased chemicals 50. Although FIG. 3 provides a general flow in the production of a desalinated plant-based bioglycerin 20, the process may instead provide a purified plant-based bioglycerin 40 for the production of biobased chemicals 50. A general flow is outlined in FIG. 3, but any of the exchange treatments may be repeated or skipped altogether, depending on the requirements and product specifications for the intended use. Additionally, FIG. 3 shows a batch flow that is first subjected to anion exchange treatments and is then subjected to cation exchange treatments. However, the cation exchange treatments may be conducted before the anion exchange treatments if the material requires this process or if the batch process desalination treatment 12 is set-up to process the high salt plant-based bioglycerin 14 with the batch cation exchange treatments first.

The batch purification method outlined in FIG. 3 shows a series of both anion and cation exchange treatments. The batch anion exchange treatment 80 may occur first. In an anion exchange resin treatment, the resin can reduce or remove halide, sulfate, and other anions that are present as impurities in the high salt plant-based bioglycerin 14 and instead replace those anions by the counterion bound to the anion exchange resin, typically hydroxide anions. A second and third anion exchange treatment, the batch anion exchange treatment 82 and the batch anion exchange treatment 84, may then occur. The purpose of second and third batch anion exchange treatments can be to further reduce the respective anion impurity levels of the product to specification for a desalinated plant-based bioglycerin 20 and/or a purified plant-based bioglycerin 40 for the production of biobased chemicals 50. Depending upon the resin, the anion exchange resin can be regenerated with an alkali base like aqueous sodium hydroxide, aqueous potassium hydroxide, or aqueous ammonia after the anion exchange treatment 82. This resin regeneration process is detailed further in FIG. 6.

The batch cation exchange treatment 90 may then occur after the anion exchange treatment(s). In a cation exchange resin treatments, the resin may reduce or remove sodium, potassium, calcium, and other cations from the impurities present in the high salt plant-based bioglycerin 14 and replace those cations by the counterion bound to the cation exchange resin, typically protons. The high salt plant-based bioglycerin 14 may then undergo a second and third cation exchange, the batch cation exchange treatment 92 and the batch cation exchange treatment 94. Like the anion exchange treatment process, the purpose of second and third batch cation exchange treatments can be to further reduce the respective cation levels of the product to specification for a desalinated plant-based bioglycerin 20 and/or a purified plant-based bioglycerin 40 for the production of biobased chemicals 50. Depending on the resin, the cation exchange resin can be regenerated with aqueous mineral acids like aqueous hydrochloric acid or aqueous sulfuric acid, as detailed further in FIG. 6.

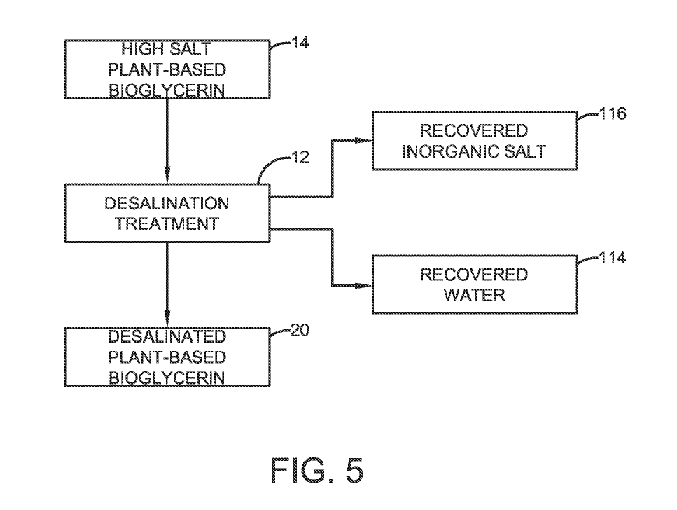

Besides the resin regeneration that provides a greener and less costly means of desalinating the high salt plant-based bioglycerin 14, the batch purification method of FIG. 3 also can potentially generate both salt and water as recoverable by-products. This process is detailed further in FIG. 5.

In FIG. 4, a detailed continuous flow purification method for the desalination treatment 12 of the high salt plant-based bioglycerin 14 is shown. During this continuous flow process, the high salt plant-based bioglycerin 14 can be converted into a desalinated plant-based bioglycerin 20. The ion exchange treatment may be done through multiple anion exchange and cation exchange treatments. These anion and cation exchange treatments typically employ an ion exchange resin to remove the negatively charged and positively charged ionic impurities present in the high salt plant-based bioglycerin 14.

First, the high salt plant-based bioglycerin 14 may be received in the continuous flow purification method for the desalination treatment 12. The high salt plant-based bioglycerin 14 can originate from a crude plant-based bioglycerin 10, a decolorized plant-based bioglycerin 30, and/or a concentrated plant-based bioglycerin 38. As the crude plant-based bioglycerin 10, the decolorized plant-based bioglycerin 30, and/or the concentrated plant-based bioglycerin 38 may be brought together as the high salt plant-based bioglycerin 14; an optional solvent addition 8 can be done. This optional solvent addition 8 can be water, an alcohol, an alcohol/water mixture, or other solvent in which the high salt plant-based bioglycerin 14 may be miscible. The optional solvent addition 8 may typically be an alcohol like methanol, but it may also be ethanol. This optional solvent addition 8 serves to reduce the viscosity of the high salt plant-based bioglycerin 14 and helps enhance recovery of the desalinated plant-based bioglycerin 20 from the ion exchange resins. Solvents used in the optional solvent addition 8 may be recovered in the concentration treatment 32, as shown in FIG. 1.

Like the batch purification method in FIG. 3, the high salt plant-based bioglycerin 14 of FIG. 4 may be subjected to multiple treatments with both anionic and cationic ion exchangers in order to produce the desalinated plant-based bioglycerin 20. The flow path for the ion exchange treatments depends upon the anion and cation level specifications for the production of either a desalinated plant-based bioglycerin 20, and/or a purified plant-based bioglycerin 40 for the production of biobased chemicals 50. Although FIG. 4 provides a general flow in the production of a desalinated plant-based bioglycerin 20, the process may instead lead to a purified plant-based bioglycerin 40 and/or be sent for the production of biobased chemicals 50.

The general flow outlined in FIG. 4 shows a continuous flow process that can be first subjected to the flow anion exchange treatments 86, and is then subjected to the flow cation exchange treatments 96. However, the cation exchange treatments may be conducted prior to the anion exchange treatments if the material requires this desalination process or if the continuous flow process desalination treatment 12 is set-up to process the high salt plant-based bioglycerin 14 with the cation exchange treatments first.

Optionally, a reduced anion plant-based bioglycerin 88 may be obtained from the flow anion exchange treatment 86, which may be subjected to one or more cycle(s) of the flow anion exchange treatment 86. These treatments are optional cycle(s) of flow anion exchange where the reduced anion plant-based bioglycerin 88 can be sent through the flow exchange column again to further reduce anion levels to the desired specifications for the production of the desalinated plant-based bioglycerin 20, and/or a purified plant-based bioglycerin 40 and/or be sent for the production of biobased chemicals 50.

Also, the reduced cation plant-based bioglycerin 98 may be optionally subjected to one or more cycle(s) of the flow cation exchange treatment 96 in order to meet the cation levels to the desired specifications for the production of the desalinated plant-based bioglycerin 20, and/or a purified plant-based bioglycerin 40 for the production of biobased chemicals 50. Like the optional cycle(s) of the flow anion exchange treatment 86 of the reduced anion plant-based bioglycerin 88, the reduced cation plant-based bioglycerin 98 can be subjected to optional cycle(s) of the flow cation exchange treatment 96.

Depending upon the resin, the anion exchange resin can be regenerated with an alkali base like aqueous sodium hydroxide, aqueous potassium hydroxide, or aqueous ammonia, and the cation exchange resin can be regenerated with aqueous mineral acids like aqueous hydrochloric acid or aqueous sulfuric acid after the continuous flow exchange process. Besides the resin regeneration providing a greener and less costly means of desalinating the high salt plant-based bioglycerin 14, the continuous flow purification method of FIG. 4 also can potentially generate both salt and water as recoverable by-products. This process is detailed further in FIGS. 5, 6 and 7.

FIG. 5 illustrates an optional water and salt recovery in the desalination treatment 12 operating under batch flow or continuous flow conditions. As the high salt plant-based bioglycerin 14 may be received, it can be subjected to the desalination treatment 12 to provide a desalinated plant-based bioglycerin 20. In the desalination treatment 12, both the recovered water 114 and the recovered inorganic salt 116 may be salvaged and used to either provide additional products or prevent additional waste streams. Besides the desalinated plant-based bioglycerin 20 and/or a purified plant-based bioglycerin 40 for the production of biobased chemicals 50, the recovered water 114 and the recovered inorganic salt 116 can be considered as additional products from the desalination treatment 12 rather than unwanted by-products or waste streams.

FIG. 6 shows the optional exchange resin regeneration 118 in the desalination treatment 12 operating under batch flow or continuous flow. After the high salt plant-based bioglycerin 14 is received, it may undergo the desalination treatment 12 to provide the desalinated plant-based bioglycerin 20. This desalination treatment 12 can use ion exchange resins to desalinate the high salt plant-based bioglycerin 14. Ion exchange resins are polymers that are capable of exchanging particular cations or anions within the polymer with ions within a solution that is passed through the ion exchange resins.

One of the advantages of using an ion exchange treatment or process to desalinate the high salt plant-based bioglycerin 14 for other applications can be that the treatment or process itself can generate little to no waste. Another advantage may be that the ion exchange resins used in the ion exchange treatment or process can be regenerated and recycled. In other words, the ion exchange resins can be used multiple times, providing a greener process with fewer waste products and minimizing costs with purchasing new ion exchange resins.

The exchange resin regeneration 118 is detailed further in FIG. 7 for the continuous flow ion exchange process.

FIG. 7 offers a depiction of the desalination treatment 12 with both the exchange resin regeneration 118 as well as the salt and water recovery while operating in a continuous flow. It also provides several optional methods to control wastes and costs associated with the ion exchange treatment or process and allows for additional products to be formed, the recovered water 114 and the recovered inorganic salt 116.

In FIG. 7, the exchange resin regeneration 118 can be used to further reduce costs and potential wastes associated with the process. One of the advantages of using an ion exchange treatment or process to desalinate the high salt plant-based bioglycerin 14 may be that the process itself generates little to no waste. Like the other green aspects of this process, the ion exchange resins used can be regenerated and recycled. In fact, the ion exchange resins can be used multiple times, providing a greener process with fewer waste products and minimizing costs with purchasing new ion exchange resins.

Ion exchange resins are polymers that are capable of exchanging particular ions within the polymer with ions within a solution that is passed through the ion exchange resins. This can occur for anion resin exchangers in the flow anion exchange treatment 86 and for cation exchange resins in the flow cation exchange treatment 96 of FIGS. 4 and 7. The ion exchange resins can be regenerated or loaded with desirable ions by washing the resin with an excess of the desired ions. The ion exchange resin can then be then flushed free of the newly-exchanged ions from desalination of the high salt plant-based bioglycerin 14 by contacting the resin with a solution of the desirable ions. Ion exchange resin regeneration may be initiated after most of the active sites on the resin have been exchanged with ions from the high salt plant-based bioglycerin 14 and the ion exchange treatment or process may no longer be effective. With the exchange resin regeneration 118, the same resin beads can be used over and over again for the flow anion exchange treatment 86 or the flow cation exchange treatment 96, and the ions that need to be removed from the system can be concentrated from the aqueous inorganic salt 112 to provide the recovered water 114 and the recovered inorganic salt 116.

There are two types of ion exchange resins used in the continuous flow process. The first may be an anion exchange resin and the second may be a cation exchange resin. Whether the anion exchange resin or the cation exchange resin may be used, the regeneration process can be similar. Although the anion exchange resin and the cation exchange resin may be processed similarly, each ion exchange resin can be separately regenerated.

After acting to desalinate the high salt plant-based bioglycerin 14 of FIG. 4, either the flow anion exchange treatment 86 or the flow cation exchange treatment 96 can be brought into the regeneration process of FIG. 7. This flow anion exchange treatment 86 or the flow cation exchange treatment 96 may consist of either the anion exchange resin or the cation exchange resin at least partially saturated with ionic impurities removed from the high salt plant-based bioglycerin 14. The anion exchange resin can then be put through the saturated anion exchange resin column 102, and the cation exchange resin may then subjected to the saturated cation exchange resin column 108. In these resin columns, the regeneration can occur. These columns can be the same or different columns from that used in the flow anion exchange treatment 86 and the flow cation exchange treatment 96.

For the anion exchange resin regeneration, typically an aqueous alkali 100 may be added to the anion exchange resin in the saturated anion exchange resin column 102. In this process, the regenerated anion exchange resin column 104 will be formed along with an aqueous inorganic salt 112. Typically, this aqueous alkali 100 can be aqueous sodium hydroxide, aqueous potassium hydroxide, aqueous ammonia, or another source of aqueous hydroxide anion that may be compatible with the anion exchange resin. From the regenerated anion exchange resin column 104, the anion exchange resin can be reused after it is directed back to the flow anion exchange treatment 86.

Alternatively in the cation exchange resin regeneration, an aqueous mineral acid 106 can be added to the cation exchange resin in the saturated cation exchange resin column 108, and the regenerated cation exchange resin column 110 may be formed along with an aqueous inorganic salt 112. Typically, this aqueous mineral acid 106 can be aqueous hydrochloric acid or aqueous sulfuric acid, with aqueous sulfuric acid being the less expensive option and could be used to keep costs down. Depending on compatibility with the cation exchange resin, certain other protic acids may be used in the regenerated cation exchange resin column 110. After this regeneration process in the regenerated cation exchange resin column 108, the cation exchange resin can be reused after it is directed back to the flow cation exchange treatment 96.

Besides the regeneration of both the anion and cation exchange resins, the process can also provide the recovered water 114 and the recovered inorganic salt 116. After both the flow anion exchange treatment 86 and the flow cation exchange treatment 96, an aqueous inorganic salt 112 may be formed in the saturated anion exchange resin column 102 and the saturated cation exchange resin column 108. Instead of initiating another waste stream, this aqueous inorganic salt 112 salt can generate yet another profitable chemical source and/or prevent disposal of another waste stream. A separation of the recovered water 114 and the recovered inorganic salt 116 can be achieved through at least one of evaporation, distillation, reverse osmosis, ion exchange, or crystallization of the salt from a saturated solution. The recovered salt may be sold for industrial applications such as road salt, chilling salts, or the like. In some cases, the salt formed during this phase may also be recovered for use as fertilizer or as a material for lowering the freezing point. Other potential applications may also include water softening, food additives, de-icing, and the production of pharmaceuticals and other chemicals.

After the water is removed from the aqueous inorganic salt 112 as the recovered water 114, it can either be safely added to the wastewater system or it could be recycled and reused elsewhere in the desalination process of FIG. 1 so as to minimize a waste stream.

FIG. 8 describes the decolorization treatment 22 that can be used in either the batch process or continuous flow process. The decolorization treatment 22 may be done on the crude plant-based bioglycerin 10, a desalinated plant-based bioglycerin 20, and/or a concentrated plant-based bioglycerin 38. At least one of the crude plant-based bioglycerin 10, the desalinated plant-based bioglycerin 20, and/or the concentrated plant-based bioglycerin 38 can be brought into the treatment as the colored plant-based bioglycerin 120.

After the colored plant-based bioglycerin 120 is collected, it may undergo an optional solvent addition 8. Like the optional solvent addition 8 in the desalination treatment 12 shown in FIGS. 3 and 4, the decolorization treatment 22 does not require this step. This optional solvent addition 8 can be water, an alcohol, an alcohol/water mixture, or other solvent in which the colored plant-based bioglycerin 120 may be miscible. The optional solvent addition 8 may typically be an alcohol like methanol, but it may also be ethanol. This optional solvent addition 8 serves to reduce the viscosity of the colored plant-based bioglycerin 120 and helps enhance recovery of the decolorized plant-based bioglycerin 30 from the charcoal treatment 122. The optional solvent addition 8 may be recovered in the concentration treatment 32, as shown in FIG. 1.

With or without the optional solvent addition 8, the colored plant-based bioglycerin 120 may then be subjected to the charcoal treatment 122. If it is used, the charcoal treatment 122 serves to reduce or remove color and improve the clarity of the resulting decolorized plant-based bioglycerin 30. The charcoal treatment 122 may work primarily by an adsorption mechanism. Adsorption is the adhesion of solid materials or dissolved materials onto a surface based on surface energy. During the charcoal treatment 122, the level of residual fatty acids and colored impurities present in the colored plant-based bioglycerin 120 can be reduced or removed by adhesion onto an adsorbent, typically activated charcoal. That is, the charcoal treatment 122 may be a more selective adsorption method for removal of these impurities than it can be for the decolorized plant-based bioglycerin 30. The colored impurities may adhere to the activated charcoal adsorbent used in the charcoal treatment 122. This charcoal treatment 122 may provide a lighter colored to a nearly clear decolorized plant-based bioglycerin 30. Depending upon the stage of the purification process of FIG. 1, the decolorized plant-based bioglycerin 30 can be over 99% pure after removal of any optionally added solvent.

Furthermore, a reduction in the level of residual fatty acid and colored impurities in the colored plant-based bioglycerin 120 can be additionally controlled depending on the number of charcoal treatment(s) 122 or process. Depending on the intended use of the decolorized plant-based bioglycerin 30, the charcoal treatment 122 may have a variety of different processing methods. These methods may include the additional step of repeating cycles of the charcoal treatment 122 of the color treated plant-based bioglycerin 124. The optional charcoal treatment 122 and the number of its repeating cycles can depend on the color of the colored plant-based bioglycerin 120 and level of the colored impurities. However, a more decolorized plant-based bioglycerin 30 may require increased energy and costs associated with additional cycles of the charcoal treatment(s) 122.

After the desired color of the color treated plant-based bioglycerin 124 may be achieved through the charcoal treatment(s) 122, the color treated plant-based bioglycerin 124 can move to a decolorized plant-based bioglycerin 30. The resulting decolorized plant-based bioglycerin 30 may be sent to the desalination treatment 12, or the concentration treatment 32, or can become a purified plant-based bioglycerin 40 for the production of biobased chemicals 50 as illustrated in FIG. 1.

In FIG. 8, the activated charcoal used in the charcoal treatment 122 can be regenerated and recycled more than one time to further reduce costs and potential wastes associated with the decolorization treatment 22 or process. This regeneration may take place whenever the adsorbent becomes saturated with impurities removed from the colored plant-based bioglycerin 120, or when the efficiency of the charcoal treatment 122 is reduced. The activated charcoal can be regenerated through an adsorbent regeneration 160 step involving at least one of steam regeneration, thermal activation regeneration, and chemical regeneration. One of the advantages of using the decolorization treatment 22 or process to decolorize the colored plant-based bioglycerin 120 may be that the process itself generates little to no waste. Like the other green aspects of this crude plant-based bioglycerin purification process, the activated charcoal adsorbent used can be regenerated and recycled. In fact, the activated charcoal adsorbent can be used more than one time to decolorize the colored plant-based bioglycerin 120, providing a greener process with fewer waste products and minimizing costs with purchasing new adsorbent.

FIG. 9 describes the concentration treatment 32 that can be used in either the batch process or continuous flow process. FIG. 9 shows the process in which a diluted plant-based bioglycerin 130 may be treated to provide the concentrated plant-based bioglycerin 38 and/or the recovered alcohol and water 134.

The concentration treatment 32 may be done on the crude plant-based bioglycerin 10, a desalinated plant-based bioglycerin 20, and/or a decolorized plant-based bioglycerin 30. At least one of the crude plant-based bioglycerin 10, the desalinated plant-based bioglycerin 20, and/or the decolorized plant-based bioglycerin 30 can be brought into the treatment as the diluted plant-based bioglycerin 130.

With the concentration treatment 32, the diluted plant-based bioglycerin 130 may undergo the evaporator/concentrator treatment 132 to produce the concentrated plant-based bioglycerin 38 and/or the recovered alcohol and water 134. In the evaporator/concentrator treatment 132, the lower boiling alcohol and water impurities can be separated from the diluted plant-based bioglycerin 130 under reduced pressure and modest temperatures. When the alcohol is methanol or ethanol, these temperatures may be about 25° C. to about 60° C. These reduced pressures may be about 20 mm Hg to about 70 mm Hg. These temperatures may also be higher or the pressures further reduced depending upon the material and equipment capabilities and requirements. By using this concentration treatment 32, the recovered alcohol and water 134 may be removed from the diluted plant-based bioglycerin 130 and the resulting concentrated plant-based bioglycerin 38 may be further processed by the desalination treatment 12, or the decolorization treatment 22, and/or be sent to a purified plant-based bioglycerin 40 for the production of biobased chemicals 50 as shown in FIG. 1.

Similarly with the concentration treatment 32, the diluted plant-based bioglycerin 130 may undergo the evaporator/concentrator treatment 132 to remove solvent from the diluted plant-based bioglycerin 130 which may be added during the desalination treatment 12 and/or the decolorization treatment 22 steps of the purification process in FIG. 1. That is to say, the concentration treatment 32 may produce the concentrated plant-based bioglycerin 38, and/or the recovered alcohol and water 134, and/or a solvent.

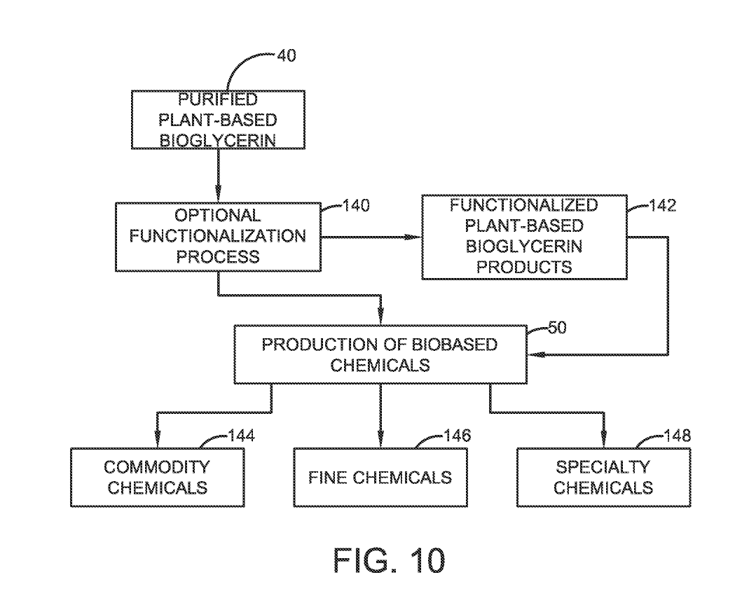

FIG. 10 shows a flowchart of several biobased chemicals, derivative products, and purified glycerin that may be formed from the process. First, the production of biobased chemicals 50 may be provided by a purified plant-based bioglycerin 40 of various purities. Alternatively, the production of biobased chemicals 50 may be provided by the crude plant-based bioglycerin 10 as shown in FIG. 1. Additionally, an optional functionalization process 140 can also be done to provide the functionalized plant-based bioglycerin products 142 and also lead further to the production of biobased chemicals 50. This optional functionalization process 140 may serve to further present added commodity chemicals 144, fine chemicals 146, and/or specialty chemicals 148 that may not be made without this functionalization. This optional functionalization process 140 may include chemical, catalytic, and/or biological means of functionalizing the purified plant-based bioglycerin 40, and/or the crude plant-based bioglycerin 10, prior to the production of biobased chemicals 50. Examples of an optional functionalization process 140 may include, but are not limited to, the preparation of 5- and 6-membered ring acetals and ketals, esterifications, and oxidations of the purified plant-based bioglycerin 40 and/or the crude plant-based bioglycerin 10 to provide the functionalized plant-based bioglycerin products 142 like glycerol formal, 4-(hydroxymethyl)-1,3-dioxolan-2-one, solketal, and glyceraldehyde.

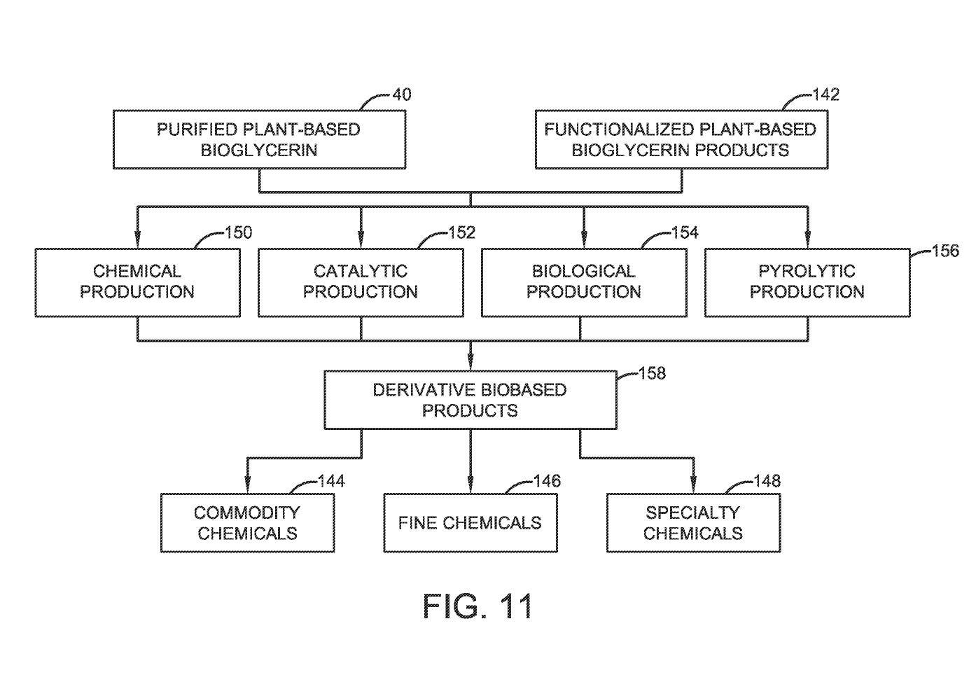

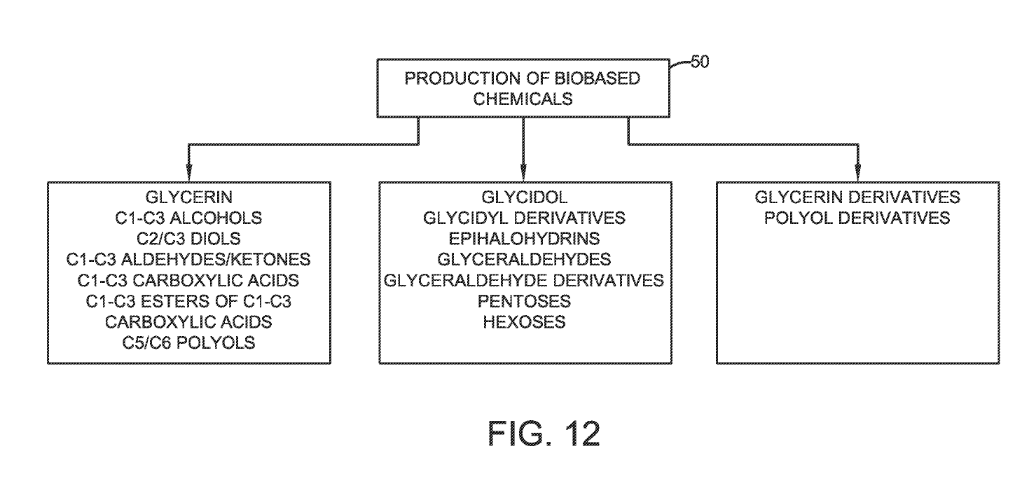

From the production of biobased chemicals 50, either with or without the optional functionalization process 140, commodity chemicals 144, fine chemicals 146, and/or specialty chemicals 148 may be produced. Several of these commodity chemicals 144, fine chemicals 146, and/or specialty chemicals 148 may be those shown in FIGS. 11 and 12.

FIG. 11 illustrates the production of biobased chemicals 50 from either the purified plant-based bioglycerin 40 and/or the functionalized plant-based bioglycerin products 142. In other instances, the production of biobased chemicals 50 may directly proceed from the crude plant-based bioglycerin 10 as shown in FIG. 1. These derivative biobased products 158 can be converted into commodity chemicals 144, fine chemicals 146, and/or specialty chemicals 148. From FIG. 11, the purified plant-based bioglycerin 40 and/or the functionalized plant-based bioglycerin products 142 may be converted into the derivative biobased products 158 through methods of chemical production 150, catalytic production 152, biological production 154, and/or pyrolytic production 156. By using at least one of the conversion methods, including chemical production 150, catalytic production 152, biological production 154, and/or pyrolytic production 156, the purified plant-based bioglycerin 40 and/or the functionalized plant-based bioglycerin products 142 may be able to produce the derivative biobased products 158 that have both financial value by conversion to value-added products and utilization of a low-value by-product/waste stream of biodiesel production 60. Additionally, the plurality of the production of biobased chemicals 50 and/or the derivative biobased products 158 produced from the functionalized plant-based bioglycerin products 142 and/or the purified plant-based bioglycerin 40 may comprise at least one of achiral, racemic, and optically pure products. These derivative biobased products 158, including commodity chemicals 144, fine chemicals 146, and/or specialty chemicals 148, may be specifically modified to provide at least one of achiral, racemic, and optically pure products. Based on the method of conversion of the purified plant-based bioglycerin 40 and/or the functionalized plant-based bioglycerin products 142 to the production of biobased chemicals 50, the derivative biobased products 158 may be selectively produced.

FIG. 12 provides some potential end products from the production of biobased chemicals 50. These end products from the production of biobased chemicals 50 may further include the production of other chemicals, materials, and products. These products may be selectively produced using the method described herein.

The product categories of the end products from the production of biobased chemicals 50 may include but are not limited to purified glycerin, glycerin derivatives, C1-C3 alcohols, C2/C3 diols, C1-C3 aldehydes/ketones, C1-C3 carboxylic acids, C1-C3 esters of C1-C3 carboxylic acids, C5/C6 polyols, polyol derivatives, glycidol, glycidyl derivatives, glyceraldehyde, glyceraldehyde derivatives, and epihalohydrins. Thereunder the production of biobased chemicals 50, a plurality of specific chemicals can be made comprising but not limited to purified glycerin, methanol, ethanol, n-propanol, isopropanol, allyl alcohol, propargyl alcohol, ethylene glycol, 1,2-propanediol, 1,3-propanediol, formaldehyde, acetaldehyde, propionaldehyde, glyoxal, acrolein, acetone, 1-hydroxyacetone, 1,3-dihydroxyacetone, formic acid, acetic acid, glycolic acid, glyoxylic acid, oxalic acid, propionic acid, lactic acid, 2,3-dihydroxypropionic acid, pyruvic acid, acrylic acid, malonic acid, hydroxymalonic acid, methyl formate, methyl acetate, methyl glycolate, methyl glyoxylate, dimethyl oxalate, methyl propionate, methyl lactate, methyl 2,3-dihydroxypropionate, methyl pyruvate, methyl acrylate, dimethyl malonate, dimethyl hydroxymalonate, ethyl formate, ethyl acetate, ethyl glycolate, ethyl glyoxylate, diethyl oxalate, ethyl propionate, ethyl lactate, ethyl 2,3-dihydroxypropionate, ethyl pyruvate, ethyl acrylate, diethyl malonate, diethyl hydroxymalonate, n-propyl formate, n-propyl acetate, n-propyl glycolate, n-propyl glyoxylate, di-n-propyl oxalate, n-propyl propionate, n-propyl lactate, n-propyl 2,3-dihydroxypropionate, n-propyl pyruvate, n-propyl acrylate, di-n-propyl malonate, di-n-propyl hydroxymalonate, isopropyl formate, isopropyl acetate, isopropyl glycolate, isopropyl glyoxylate, diisopropyl oxalate, isopropyl propionate, isopropyl lactate, isopropyl 2,3-dihydroxypropionate, isopropyl pyruvate, isopropyl acrylate, diisopropyl malonate, diisopropyl hydroxymalonate, allyl formate, allyl acetate, allyl glycolate, allyl glyoxylate, diallyl oxalate, allyl propionate, allyl lactate, allyl 2,3-dihydroxypropionate, allyl pyruvate, allyl acrylate, diallyl malonate, diallyl hydroxymalonate, glycerol formal, 4-(hydroxymethyl)-1,3-dioxolan-2-one, 4-methyl-1,3-dioxolane, (2,2-dimethyl-1,3-dioxolan-4-yl)methanol, 1,4-dioxaspiro[4.5]decane-2-methanol, glyceraldehyde, 2,2-dimethyl-1,3-dioxolane-4-carbaldehyde, 1,4-dioxaspiro[4.5]decane-2-carbaldehyde, glycidol, glycidyl methyl ether, glycidyl ethyl ether, glycidyl n-propyl ether, glycidyl isopropyl ether, glycidyl n-butyl ether, glycidyl isobutyl ether, glycidyl sec-butyl ether, glycidyl tert-butyl ether, glycidyl allyl ether, glycidyl propargyl ether, glycidyl hexadecyl ether, glycidyl octyl/decyl ether, glycidyl phenyl ether, glycidyl benzyl ether, glycidyl formate, glycidyl acetate, glycidyl propionate, glycidyl isopropionate, glycidyl n-butyrate, glycidyl isobutyrate, glycidyl sec-butyrate, glycidyl acrylate, glycidyl methacrylate, diglycidyl 1,2-cyclohexanedicarboxylate, glycidyl benzoate, glycidyl 4-nitrobenzoate, epichlorohydrin, epibromohydrin, ribitol, arabitol, xylitol, mannitol, sorbitol, galactitol, allitol, iditol, and bis-(2,2-dimethyl-(1,3)dioxolan-4-yl methanol. This production of biobased chemicals 50 as described herein can allow for both the utilization of a renewable, carbonaceous by-product in the production of value-added chemicals and biobased products and an even greener biodiesel production 60 process.

Having thus described the invention, it is now claimed:

I/we claim:

1. A method of biorefining, comprising the steps of:
providing plant-based bioglycerin;
treating said plant-based bioglycerin to provide treated plant-based bioglycerin; and
producing at least one derivative product from at least one of said plant-based bioglycerin and said treated plant-based bioglycerin.

2. The method of claim 1, further comprising the step of:
providing said plant-based bioglycerin as a by-product of biodiesel production.

3. The method of claim 1, wherein said plant-based bioglycerin is provided from at least one plant-based triglyceride of soybean oil, corn oil, cottonseed oil, canola oil, rice bran oil, flax oil, sunflower oil, safflower oil, artichoke oil, sesame oil, peanut oil, castor oil, coconut oil, colza oil, false flax oil, hemp oil, mustard oil, palm oil, radish oil, rapeseed oil, tigernut oil, tung oil, copaiba oil, jatropha oil, jojoba oil, karanj oil, milk bush (pencil bush) oil, neem oil, olive oil, salicornia oil, and paradise oil.

4. The method of claim 1, wherein the step of treating said plant-based bioglycerin to provide said treated plant-based bioglycerin comprises at least one treatment of desalination treatment, decolorization treatment, and concentration treatment.

5. The method of claim 4, wherein said desalination treatment provides desalinated plant-based bioglycerin as said treated plant-based bioglycerin.

6. The method of claim 4, wherein said decolorization treatment provides decolorized plant-based bioglycerin as said treated plant-based bioglycerin.

7. The method of claim 4, wherein said concentration treatment provides concentrated plant-based bioglycerin as said treated plant-based bioglycerin.

8. The method of claim 4, wherein the step of treating said plant-based bioglycerin to provide desalinated plant-based bioglycerin, further comprises the step of:
utilizing a desalination treatment.

9. The method of claim 4, further comprising the step of recovering salt.

10. The method of claim 9, further comprising the step of:
lowering a freezing point of an aqueous solution with said salt.

11. The method of claim 4, wherein the step of treating said plant-based bioglycerin to provide desalinated plant-based bioglycerin, further comprises the step of:
adding a solvent.

12. The method of claim 8, wherein the step of treating said plant-based bioglycerin to provide said desalinated plant-based bioglycerin, further comprises the step of:
utilizing an ion exchange treatment.

13. The method of claim 12, further comprising the step of:
performing said ion exchange treatment under a batch condition.

14. The method of claim 12, further comprising the step of:
performing said ion exchange treatment under a continuous flow condition.

15. The method of claim 12, further comprising the steps of:
recovering water during said ion exchange treatment; and
recycling said water during said ion exchange treatment.

16. The method of claim 12, further comprising the steps of:
recovering a solvent during said ion exchange treatment; and
recycling said solvent during said ion exchange treatment.

17. The method of claim 12, further comprising the steps of:
regenerating an ion exchange resin during said ion exchange treatment; and
recycling said ion exchange resin during said ion exchange treatment.

18. The method of claim 4, wherein the step of treating said plant-based bioglycerin to provide decolorized plant-based bioglycerin, further comprises the step of:
utilizing a decolorization treatment.

19. The method of claim 4, wherein the step of treating said plant-based bioglycerin to provide decolorized plant-based bioglycerin, further comprises the step of:
adding a solvent.

20. The method of claim 6, further comprising the step of:
utilizing activated charcoal in said decolorization treatment.

21. The method of claim 6, further comprising the steps of:
recovering a solvent during said decolorization treatment; and
recycling said solvent during said decolorization treatment.

22. The method of claim 6, further comprising the step of:
performing said decolorization treatment under at least one condition of a batch condition and a continuous flow condition.

23. The method of claim 20, further comprising the steps of:
recovering a solvent during said decolorization treatment with said activated charcoal; and
recycling said solvent during said decolorization treatment with said activated charcoal.

24. The method of claim 20, further comprising the steps of:
regenerating said activated charcoal during said decolorization treatment; and
recycling said activated charcoal during said decolorization treatment.

25. The method of claim 4, wherein the step of treating said plant-based bioglycerin to provide concentrated plant-based bioglycerin, further comprises the step of:
utilizing a concentration treatment.

26. The method of claim 25, further comprising the step of:
utilizing an evaporation or distillation process during said concentration treatment.

27. The method of claim 26, further comprising the steps of:
recovering water during said concentration treatment; and
recycling said water during said concentration treatment.

28. The method of claim 26, further comprising the steps of:
recovering a solvent during said concentration treatment; and
recycling said solvent during said concentration treatment.

29. The method of claim 26, further comprising the step of:
performing said concentration treatment under a batch condition or a continuous flow condition.

30. The method of claim 1, wherein said plant-based bioglycerin has a weight, said treated plant-based bioglycerin has a weight, and said weight of treated plant-based bioglycerin is greater than about 60% of said weight of plant-based bioglycerin.

31. The method of claim 1, wherein said plant-based bioglycerin has a weight, said treated plant-based bioglycerin has a weight, and said weight of treated plant-based bioglycerin is greater than about 80% of said weight of plant-based bioglycerin.

32. The method of claim 1, wherein said plant-based bioglycerin has a weight, said treated plant-based bioglycerin has a weight, and said weight of treated plant-based bioglycerin is greater than about 90% of said weight of plant-based bioglycerin.

33. The method of claim 1, further comprising the step of selectively producing said derivative product from said plant-based bioglycerin and said treated plant-based bioglycerin.

34. The method of claim 1, wherein said producing at least one of said derivative product comprises at least one chemical of commodity chemicals, fine chemicals, and specialty chemicals.

35. The method of claim 1, wherein said producing at least one of said derivative product comprises at least one process of a chemical process, a biological process, a catalytic process, and a pyrolytic process.

36. The method of claim 1, further comprising the step of functionalizing said plant-based bioglycerin or said treated plant-based bioglycerin prior to production of at least one of said derivative product.

37. The method of claim 1, wherein at least one of said derivative products comprise purified glycerin, glycerin derivatives, C1-C3 alcohols, C2/C3 diols, C1-C3 aldehydes/ketones, C1-C3 carboxylic acids, C1-C3 esters of C1-C3 carboxylic acids, C5/C6 polyols, polyol derivatives, glycidol, glycidyl derivatives, glyceraldehyde, glyceraldehyde derivatives, and epihalohydrins produced from the said plant-based bioglycerin or said treated plant-based bioglycerin.

38. The method of claim 37, wherein at least one of said C1-C3 alcohols comprise methanol, ethanol, n-propanol, isopropanol, allyl alcohol, and propargyl alcohol.

39. The method of claim 37, wherein at least one of said C2/C3 diols comprise ethylene glycol, 1,2-propanediol, and 1,3-propanediol.

40. The method of claim 37, wherein at least one of said C1-C3 aldehydes/ketones comprise formaldehyde, acetaldehyde, propionaldehyde, glyoxal, acrolein, acetone, 1-hydroxyacetone, and 1,3-dihydroxyacetone.

41. The method of claim 37, wherein at least one of said C1-C3 carboxylic acids comprise formic acid, acetic acid, glycolic acid, glyoxylic acid, oxalic acid, propionic acid, lactic acid, 2,3-dihydroxypropionic acid, pyruvic acid, acrylic acid, malonic acid, and hydroxymalonic acid.

42. The method of claim 37, wherein at least one of said C1-C3 esters of C1-C3 carboxylic acids comprise methyl formate, methyl acetate, methyl glycolate, methyl glyoxylate, dimethyl oxalate, methyl propionate, methyl lactate, methyl 2,3-dihydroxypropionate, methyl pyruvate, methyl acrylate, dimethyl malonate, dimethyl hydroxymalonate, ethyl formate, ethyl acetate, ethyl glycolate, ethyl glyoxylate, diethyl oxalate, ethyl propionate, ethyl lactate, ethyl 2,3-dihydroxypropionate, ethyl pyruvate, ethyl acrylate, diethyl malonate, diethyl hydroxymalonate, n-propyl formate, n-propyl acetate, n-propyl glycolate, n-propyl glyoxylate, di-n-propyl oxalate, n-propyl propionate, n-propyl lactate, n-propyl 2,3-dihydroxypropionate, n-propyl pyruvate, n-propyl acrylate, di-n-propyl malonate, di-n-propyl hydroxymalonate, isopropyl formate, isopropyl acetate, isopropyl glycolate, isopropyl glyoxylate, diisopropyl oxalate, isopropyl propionate, isopropyl lactate, isopropyl 2,3-dihydroxypropionate, isopropyl pyruvate, isopropyl acrylate, diisopropyl malonate, diisopropyl hydroxymalonate, allyl formate, allyl acetate, allyl glycolate, allyl glyoxylate, diallyl oxalate, allyl propionate, allyl lactate, allyl 2,3-dihydroxypropionate, allyl pyruvate, allyl acrylate, diallyl malonate, and diallyl hydroxymalonate.

43. The method of claim 37, wherein at least one of said purified glycerin and glycerin derivatives comprise purified glycerin, glycerol formal, 4-(hydroxymethyl)-1,3-dioxolan-2-one, 4-methyl-1,3-dioxolane, (2,2-dimethyl-1,3-dioxolan-4-yl)methanol, and 1,4-dioxaspiro[4.5]decane-2-methanol.

44. The method of claim 37, wherein at least one of said glyceraldehyde and glyceraldehyde derivatives comprise glyceraldehyde, 2,2-dimethyl-1,3-dioxolane-4-carbaldehyde, and 1,4-dioxaspiro[4.5]decane-2-carbaldehyde.

45. The method of claim 37, wherein at least one of said glycidol and glycidyl derivatives comprise glycidol, glycidyl methyl ether, glycidyl ethyl ether, glycidyl n-propyl ether, glycidyl isopropyl ether, glycidyl n-butyl ether, glycidyl isobutyl ether, glycidyl sec-butyl ether, glycidyl tert-butyl ether, glycidyl allyl ether, glycidyl propargyl ether, glycidyl hexadecyl ether, glycidyl octyl/decyl ether, glycidyl phenyl ether, glycidyl benzyl ether, glycidyl formate, glycidyl acetate, glycidyl propionate, glycidyl isopropionate, glycidyl n-butyrate, glycidyl isobutyrate, glycidyl sec-butyrate, glycidyl acrylate, glycidyl methacrylate, diglycidyl 1,2-cyclohexanedicarboxylate, glycidyl benzoate, and glycidyl 4-nitrobenzoate.

46. The method of claim 37, wherein at least one of said epihalohydrins comprise epichlorohydrin and epibromohydrin.

47. The method of claim 37, wherein at least one of said polyols and polyol derivatives comprise ribitol, arabitol, xylitol, mannitol, sorbitol, galactitol, allitol, iditol, and bis-(2,2-dimethyl-(1,3)dioxolan-4-yl methanol.

48. The method of claim 1, wherein at least one of said derivative products comprise achiral, racemic, and optically pure products.

49. The method of claim 1, further comprising the step of:
using at least one of said derivative product in the production of other chemicals, materials, and products.

50. The method of claim 5, wherein said desalinated plant-based bioglycerin has a weight, and a waste product of said desalinated plant-based bioglycerin is less than 60% of said desalinated plant-based bioglycerin weight.

51. The method of claim 6, wherein said decolorized plant-based bioglycerin has a weight, and a waste product of said decolorized plant-based bioglycerin is less than 60% of said decolorized plant-based bioglycerin weight.

52. The method of claim 7, wherein said concentrated plant-based bioglycerin has a weight, and a waste product of said concentrated plant-based bioglycerin is less than 60% of said concentrated plant-based bioglycerin weight.

53. A method for biorefining, comprising the steps of:
providing plant-based bioglycerin;
treating said plant-based bioglycerin to provide treated plant-based bioglycerin; and
using waste product from said treated plant-based bioglycerin to produce energy.

54. The method of claim 53, wherein said energy is heat or power.

55. A method of biorefining, comprising the steps of:
providing plant-based bioglycerin as a by-product of biodiesel production;
providing said plant-based bioglycerin from at least one plant-based triglyceride of soybean oil, corn oil, cottonseed oil, canola oil, rice bran oil, flax oil, sunflower oil, safflower oil, artichoke oil, sesame oil, peanut oil, castor oil, coconut oil, colza oil, false flax oil, hemp oil, mustard oil, palm oil, radish oil, rapeseed oil, tigernut oil, tung oil, copaiba oil, jatropha oil, jojoba oil, karanj oil, milk bush (pencil bush) oil, neem oil, olive oil, salicornia oil, and paradise oil;
treating said plant-based bioglycerin to provide treated plant-based bioglycerin comprising at least one treatment of desalination treatment, decolorization treatment, and concentration treatment;
treating said plant-based bioglycerin to provide desalinated plant-based bioglycerin using an ion exchange treatment in at least one condition of a batch flow condition and continuous flow condition;
treating said plant-based bioglycerin to provide decolorized plant-based bioglycerin using activated charcoal in at least one of said condition of a batch flow condition and continuous flow condition;
treating said plant-based bioglycerin with said activated charcoal;
treating said plant-based bioglycerin to provide concentrated plant-based bioglycerin using at least one of an evaporation process and distillation process in at least one of said condition of a batch flow condition and continuous flow condition;
producing at least one derivative product from said plant-based bioglycerin and said treated plant-based bioglycerin by at least one process of a chemical process, biological process, catalytic process, and pyrolytic process;
recovering and recycling said water, said solvent, and said ion exchange resin from said desalination process;
recovering said salt from said desalination process;
recovering and recycling said solvent from said decolorization process;
recovering and recycling said activated charcoal from said decolorization process;
recovering and recycling said water from said concentration process;
recovering and recycling said solvent from said concentration process;
recovering said treated plant-based bioglycerin, wherein said plant-based bioglycerin has a weight, said treated plant-based bioglycerin has a weight, and said weight of treated plant-based bioglycerin is greater than 80% of said weight of plant-based bioglycerin;
reducing a waste product of said treated plant-based bioglycerin, wherein said treated plant-based bioglycerin has a weight, and said waste product of said treated plant-based bioglycerin is less than 60% of said desalinated plant-based bioglycerin weight;
producing energy from said waste product of said treated plant-based bioglycerin;
functionalizing said plant-based bioglycerin and said treated plant-based bioglycerin prior to production of at least one of said derivative product; and
producing at least one of said derivative product comprising purified glycerin and glycerin derivatives, C1-C3 alcohols, C2/C3 diols, C1-C3 aldehydes/ketone, C1-C3 carboxylic acids, C1-C3 esters of C1-C3 carboxylic acids, C5/C6 polyols, polyol derivatives, glycidol, glycidyl derivatives, glyceraldehyde, glyceraldehyde derivatives, and epihalohydrins from the said plant-based bioglycerin and said treated plant-based bioglycerin.
